SEQUENCE LISTING (20 sequences)

SEQ ID NO: 1 - DNA, 870 bp, Unknown
  Description of Unknown Bacterial polynucleotide
atgtacattgcagatggaaaaacaaacggaccagcgttttcctggccagacggcaaacgcattgccgtgatggttacatttgattatgacgctgaatttttacggatatcccgcgccaaaagcaagggaacgagcattggctttaccgatttttcacgaggccagtacggcccccatgagggactggccagatgccttgatatgttagataccatgaacatcaaatctaccttttttgtgccgggcgctgtgatcgagacctaccgggataccgtagaggaaatccaccggcgcggccatgagctggcctgccacggctaccggcatgaatccgatccggagctttcccgggacgaaatgataaaaatcctggataaaagtgaagcgctgctcgcagagatcaccggaaaaaagccagtaggacaccgggcaccggaaagcgtgctccaggattttatgccggagcttctggctgagcgtggctatctgtacagttcttccatgaaggactgtgactgggcttatctctgggaaaaggatcaaaaggagctgcccctggtagagctcccaaacgatatcaccatggatgactttacctattactatttcaccttcagtgatcctgcagtccgctgtatgtacccgaaccgtgaggtcttcggcaactggaagcaggaatttgacggtctggccctggagggcaacaagatcttcatcttaaagctgcatccgcagatgatcggccgcgcgagccgcatcggcatggtaggcgaattcattgcctacatgcagaatcacggcgcatggatcaccacctgcgaggatgtagcacgttatgtacagaagcagaacggaggaaacagagcatga

SEQ ID NO: 2 - DNA, 1170 bp, Unknown
  Description of Unknown Bacterial polynucleotide
atgatccggaaacgtgcaaaacggctggcaggcgcagtgcttgccgccggcgccattacggcaatgccatttcaggcatttgcccagagaagcccggaatttgcttattcagcagaaaaatgggcgacactccgcgacaataagctggagtttgatgagatttcagatctggttcatgagtacaatccgaccgtggtccagaacgagatcagctacaaggattatctgaccaagaaccgggatgatgtggcccaggattattacgacaaggccaatgagatttattccaatatcagctacccggactcagatgatgccaactacggaagtggtgtggcagcggcactgcgcaatgaacagcaggccaaaagcctgatggagcagggtgatgaaaacaccgatgaccaggccaccatgcggatccagtacgatcaggccgaggcgaagctggccaagcaggcacaggggcttatgatcacctactggacccagtactataacctggatggccagaaggcccgcgttgagcaggcgaagctttcgtaccagtctgagcagaaccgcctggcagcagggatgtctacccagtccaaggttttaagcgcaaaggagtccgtctccaatgcggaggcagcgctggtgactgccgagagcaatctggcctccacaaaggagagtctgtgcctgatgttaggctggggctacggcgcggatgtggagatcgcggagcttgcagaaccggaccagagtaagatcgcggccattgatgtgaatgcggatatccaggcagctctggagaacagctacgcctaccgcttgacgaaaaagcaactcaccaacgccagaacagacagcgtgaaggataagctgagcgagacggaaaagaatcagagggagaccatctccaacagtgtgaaatctgcttatgattccctgcttctggctcagtccggttacgagcaggctcagtccgcgctggcgctgcaggaggtttccatgaagtctgtcgacgcgaagctggcagcgggaaccattacaaaaaatacctatgagagccagaaggcatcctacaccaccgcccaggtgactgcccagacccagaagctgtccctgttacaggccatgaatgattatgactgggccgtgaacggactggcatctgcagagtaa

SEQ ID NO: 3 - DNA, 987 bp, Unknown
  Description of Unknown Bacterial polynucleotide
atggcagagaatattttacaggtaaaaaatttaaaaacctactttcatactgaggccggacttgtgaaagcggttaacgatgtttccttcaatgtggaaaagggtaagaccctcggcattgtaggtgagtccggctgcggaaaaagtatcacttccttatcgatcatgggtctggtagagcgtcccggtaagatcgagggcggtgagatcctgtttgagggggaagacctcttaaagatgacggaggctcagatgcgcaacatccgcggcaagaagattgccatgatcttccaggagccgatgacatccttgaatccggtttataccatcggacagcagctgatcgaggccctgctccttcatgagaaaatgaccaagcaggaagcaaaagcccgcgccatcgagatgttaaagctggttaagatcccgcttgcggagcgccgcttcgacgagtacccgcatcagctttccggcggtatgcgtcagcgtgtcatgattgccatggcactctgctgtaatccggctatgctgatctgtgatgagccgactactgcgctggacgtaaccattcaggcgcagattctggacctcatcaatgagttaaaagaaaagaccggaacctccgttatgatgattacccacgacctgggtgtcatcgcagaggttgcggatgatgttatggttatgtacgcaggcaaggtcgttgagcacgcaagctgcgaccagatctttgacaagccgatgcatccgtatacggacggactgatgaagtgcattccgaagctggatgatgacgacaccaaagagctgagcgttatcaagggcatggtgccaagctttgatgatatgccggcaggctgcgcgttctgcccgagatgtccgcaggcccgcgagatctgccgtcagaagatgccggagttagtcgaagccgagggccgcaaagtgcgctgctttaaatatacgaaggagtgggaggagaacatctga

SEQ ID NO: 4 - DNA, 576 bp, Unknown
  Description of Unknown Bacterial polynucleotide
atggagcttttaaaacagcggatcctgcgggacgggcaggtgaaagaaggcggcgtactcaaggtggacagctttctcaaccaccaaatggatgtgacgctgctgaatgagatcgggcgtgaattccgccgccgtttcgacggagcggccatcaccaaaatcgttaccatcgaggcgtccggcatcggtattgccgccattgccgcgcagcatttcggcagcgttcccgtcatcttcgccaaaaagacgcgttcgcgcaatctggacggtgcgctgtataccgccaaggtccattctttcaccaaggacattacctacgatgtgcagctatccaaaaaatttctcggcccgcaggacactgtgctcatcctggacgatttcctcgcccggggacaggcgctgctgggcctcatcgacatcgtgcggcaggcgggcgcgcagtgcgctggctgcggcatcgtcattgaaaagcagcagcagggcggcggcgcgctggtgcgggcagcgggagtgcgcctggaatcgctggccgtaatcgcctcgctggaaaacggcacggtcacctttgcgcagtga

SEQ ID NO: 5 - DNA, 1758 bp, Clostridium hathewayi
  DSM 13479
atggcggactgccctattaactcagcctcagagactattgctgactttatctaccgtaatggaagccagcagcaatttcctggtaattcggaagatattccctgtatggactttgtaagcactgatttcagcattatatataccccgctagataccgtagaacccatatcactaagtaagtttacctattatagtattcctggactgtacacactgttagatagttccagcatggacgcttccggcattttggcaacccatgcatcacctgcccttagcaatcaagggagaggtgttataatcggcatcatagacacgggaattgactataccaatcctttatttcgtaatcaggatggaactaccagaatcttgagtatttgggatcagagccttccagaagataaaagcctccttcctgccggtgtacccaaccgctataatgccagcggggcaagctatggcacagagtacacgcaggagcagattaacgaggcactggaatctgataatccgtttgccgtggtgccttctaccgataccaacggacatggaacctttttggcgggtatcgctgctggaggaatcctgcctaatcaggattttacaggagcagcaccggaatgtgaactggtaatcgtcaaattaaaaccagccaaacaatatctgcgcgatttctatcttatcagtaatgatgcggatgcttatcaggagaatgatataatgatgggcattaagtacctgcgtgtggaggcatacagccagagaaaacctctggttattttactgggaatcggctccaatctgggcagccatgaagggacgtctccattgaatgtaatgattcaagatatcagccgatatttaggtatggctacggtaattgctgcaggcaatgaaactggccgggggcatcattacatggggtctgtaccgataggggaagagtccgttgaggtggaaatccgagtagggaatgcggaatcccaacgtggatttgtagtagaactctgggccgacaccgctgatacatattcggttggatttgtctcacccagtggtgaatccataggccgcattcctatcataggcagaaacgaaacctctatcccgttccttttggaacccacggtcattacagttaattaccagctgattgagacaggcgcaggcaaacagcttgtttttatccgttttgcagccccaactaatggaatttggagaatacgcgtgtataatacccagtatcttacagggcaatttaatatgtggcttccggcccataccttgatttctgatgaaaccgtgtttctgactcccagcccatacacgaccataaccctcccgggagattctccctctccaatcacagtaggagcctataatcatttaaataacagcatctacattcattccggccgtggatatacaataggcggattgataaagccggatttggcagctccaggggttaatgtaagcgggccatcgatagggcaaaggaaccaagactccattcccatgactacccgaaccggaacctcggttgctgctgcccatgttgctggtgcagtcgctaatcttttaagctggggcataatagaaggacataacatagcaatgagcgaagccactgtcaaagcatttttaataagaggagcaaaacggaatccagcactatcatatcccaatagggaatggggatatggggctctgaacttatatgaaacttttttacggttaagagaaataagatga

SEQ ID NO: 6 - DNA, 1308 bp, Unknown
  Description of Unknown Bacterial polynucleotide
ttggattacttagtattaacgaaggttcatggggcgattcttggtccgatctcccagattctgggatggatcctgaatgtactcgttacgttcaccaattcctttggtgtacttaacatcggtttatccattattttatttaccctggttgtgaaattactgatgttccctatgacgatcaagcagcagaaggcatccaagctgatggctgtcatgcagcctgagcttcaggctattcaggcaaagtataagggcaaaacagacaatgactccatgatgaagatgaacgtggagacaaaagctgtctatgagaaatacggcacatccatgactggaggatgtctccagcttgtgatccagatgccgatcctgtttgcgctgtaccgcgtgatctattgcattccggcttacgtgatgccggtaaaggaacattttctgaatgtggtctatgcactgaccggaacttcttccgcgtctgccctgagcgagggggctgcggcaaatctgctccagtttgcgaccgaccacaatattgcgttaacgggcattaacccgatcggagacttaaccggtgtgaacggtgaggctctggccaataagatggttgatattctctacaagctgaatccggctcagtggaatgatctggcgcaggcattcccgaatgcggcagatgtcatttccacaaatgcgtctaccattgagaagatgaataccttccttggcattaatctggcatccaacccgtttaacggaagctttgtaccgagtctggcatggctgattccgattctggcaggtctgacccagtatgcgagcacaaagttaatgatggcaacccagccgaagaagaacaatgaggaagatatgagctcccagatgatgcagagcatgaacgttaccatgccgctgatgtctgtatttttctgctttaccttcccggcagcaatcggtatctactgggtagccagcagtaccttccagttactgcagcagcttgttgtaaattcttacctggataaggtagatatggatgagctgatccgtaagaacgttgagaaggctaacaagaaacgcgcaaagaaaggccttccgccgcagaaagtaaatcagaacgcgaccgcaagcttaaagcatatgcaggcagttcaggagaaggaagaagcggaacgcgctgagaagcttgagaagagcaaaaaacaggtagaagcatccaacaattattataataaagatgcaaaaccgggcagtctggcctccaaggcaaatatggtagccagatataatgaaaagcacaataataaataa

SEQ ID NO: 7 - DNA, 375 bp, Unknown
  Description of Unknown Bacterial polynucleotide
atggaaaaaattcacacggaaaaggccccggccgcaatcggcccttattcgcaggcgatgaagcagggggggctggtgttcacctccggccagatcccgctggacccggcgacgggcgccgtcgtgggggaagatatcacagcgcaggcaaagcaggtgcttgaaaacctcaaggccgtgctggaagcatccggtgcggcgctggatacagtgctcaagacgacatgctttttggcggatatggcggacttcgcgcccttcaacgagctgtatgcggcctatttcacaggctgcccggcacgctcctgcttggcggtgaaacagctgcccaaaggtgtgctcgtagaagtggaggccattgcggcggtgcgctga

SEQ ID NO: 8 - DNA, 396 bp, Unknown
  Description of Unknown Bacterial polynucleotide in Clostridiales family with strain number SS3/4
atgaaaaattatacaatcacagtaaacggtaatgtatatgaagtaacagtagaagagggcttcacaggcgcagcttccgcaccgaaggcagcagcaccggcaccgaaggcagcaccggcaacagcaccgaaggcagctccggcaccggcagcagctccggcagctccggctggcgcagcaggcgcagtagcagttacagctccgatgccgggtaagatccttggcgttaaggcatctgcaggtcaggctgttaagagaggtcaggttctcctgatccttgaagctatgaagatggagaacgagatcgttgctccgcaggacggcactgttgcaacaatcaacgttgctgttggtgattccgtagagccgggtgctacactcgctacattaaactaa

SEQ ID NO: 9 - DNA, 819 bp, Unknown
  Description of Unknown Bacterial polynucleotide
tctgtatttcaagttgctttccacagctttcgctgttacaataagcctatcgaagaaacggaggtcctcatgaagatcattttcgtctggctccttgccattgttttcctcatcgacagcgtcctgcgcgccatccgctccagcttcaaccttggggtgctgatgatgtatctcatcactgccgcactgtggatatacgctcttttccataccaaaatcgacgctttttgcgctgccggtgcaggccgcgttctaaaaatcatctttttctgctgctgcgccgtttttgcgctgctgcttatattcgtcgcggtgagcggctactccgacaccgcgaccaagcaggaaaaagcggtgatcgtactgggcgccggcctgcgaggcgaacgcgtgaccgaccttttggcacgccgtctggatgccgcatatgattatcatctggaaaacccgaatgccgttattgttgtgactggcggacagggtcccggcgaggacattcccgaagcccgggccatgaaagcctatcttgtggagaaaggcgtgccggagaagcaaattctggaagaagcgtccagtacctccaccgaggaaaatttttgctttgcgcgcgaaattttggaacagcacggtctttcgcaggacgaacccgtcgcgtatgtcaccaatgcgttccattgttaccgcgcagcaaaatacgctgccgccgcaggcttcacaaatgtgaacgccacccccgcctctatcggcttttcttccgtactcccctgttatatgcgtgaggtaatggcggtgctgtactactggatgtttcgcacctga

SEQ ID NO: 10 - DNA, 213 bp, Unknown
  Description of Unknown Bacterial polynucleotide
tccagcgaacggaaggatttcatcattgtttccgagcgcagaccatatccgatgaacccgatgcgtttcataattccattcctccattttttgtgcggggccgcttttctttatattacatataaaaggcgggcaaatggctttgcagtgcgcctgcgccgctttttatacgctcgctgcgctaataattattttgttattagcaaattttaa

SEQ ID NO: 11 - DNA, 1284 bp, Clostridium hathewayi
  DSM 13479
atgataattgatttgagccttcagcagtatgtggattgggaagtaaagccagtgattcctataaagggaaaattcggataccgtgttgtgttaaaatacatagatggtactgaaagaactcagcaaaagtcggggtttaggacagaaaaagagtctaatgctgccagggataacacaataggcaaattacatgctggaacttatattgtatatgagaatgttaaagtaagtgactttttagagttttggctaaaggaggatatatgcaagagggtaagaagcgaagaaacttatgctgcttattccaatattgtgtataaccacattattccaatccttggaaagaaaaagatgtcggccgttaaccgtggagatgttcaaaagctgtataacgaccgggcagaatattctgtatcggtctcaaggctggttaaaacggtgatgaatgtctctatgaactatgctgtagcgaaaaaaatcattgcggagaatccggcagttggtatcaatcttcctaaaacggtaaagaagaaggaataccatgcccgcagtattgacacacagaaaaccctgacaatggatcagattctgattctattggaggccagcagggacacgccgatacatatgcaaatcctgcttaatgtattaatgggacttcgcagaagggaaatcaacggtgtgaaatattcggatattgactatattaaccgaacactaaagcttcgccggcagttggggaagaaaatcaacacaaaaaaagaagactttccacctaaaacctttacgaaacaggaacttggattgaagaccccatcgagctatcgtgatattccgataccagattatgtgtttgaggcgattctgcaacagagggaggtgtatgagagaaataagagtcgcaggagaagccagtttcaggattcaggatatgtttgctgctctaactacgggaaaccgcgaagcaaggatttccattggaaatattataaaaaactgttagcggataacaacctgccggatataaagtggcatcatttacgcagtaccttctgtacactgcttttgaaaaataattttaatcccaaagcggtatccaaattaatgggacatgcaacggagctaatcaccttagatgtatatggtgataaccgggaaattattgctgactgtgtagatgaaattcagccgttcatagatgaggtgcttccgataaaggaggtagacaagcagcttgaggaagaactgttggaaatcgaagttccggcagaagattatgtttaa

SEQ ID NO: 12 - DNA, 180 bp, Unknown
  Description of Unknown Bacterial polynucleotide
tttccgaaagggaaggactctccaaagcgtcgtaccttgaaaactgaacaaagactgcgtaaagacaaaaggatggatgttccaccaggaagcattcatcatttgaaagagattttaaaagaaatctacaatttcaaagttcttttgttggcaaatggaagaatcgagcaagaaagataa

SEQ ID NO: 13 - DNA, 966 bp, Clostridium hathewayi
  DSM 13479
ttgctaaaaaaagaaaaggagaaagaaaaaatgaacatggaaacgtgtaagacaagttttgaagagaagttaaaggagaacttgggagcggaatataaagcgacatttccgcagcgaatggaaaaacaggacgaggaatttctcagggttggagtccaaaaatccggtgaatcagctggaatttggatatacctaaacggcagtgagtttcattggctggataatgaggaagacattcaaagggcggtaacaaaagtgatagaagagtacaaaaaaaagagaggattcttaaatggtccatatctcacaggagatagctttagcaacgtaaaaaacaatcttgtttgtgcattggccaatcgcgaagacaatgaggaagtgctgaagtatataccgcatatcagatactacgacatggtggcgtacttcttgatttttattacggtggatggagaatcgtatgttagaaccgtggtaaacgaggatttagaccggtgggaagtggagctgcaggacgtgctggaaacggcaaagtcaaatacagatctcaatccaccggtgattgaaatggttgaaattaaggactggaaggtgatatcagctccaataggaaacacatttggtgagatgctccaatctatcaaaagccatcaggaacagaagaaccctatgtatgtgctgtcaaatcaaagtggcctgtatggagccagcaatatgattgacaatcatctgttgggacagatttcagagagctgtaatgacagcctccttatacttccggtagacattcatgagattatattaattccgtccagaaaaaatagaacctctattgcggattggaaagctgtgatgcatgccttaaatattgaaaataaaggagtgaagctttctgaccttgtatatttgtttgaccgggcggataagaaactgcatgtagcaaaaaatgatgattttcaggcataa

SEQ ID NO: 14 - DNA, 11649 bp, Unknown
  Description of Unknown Bacterial polynucleotide
atgttagtctgtgccggttttttggaaatgaggaaaaatatgatacgacacgatagctttttcaaaagactgaagcgtggtggctcgctgtttttagcggtgttacttgcggttcagtctttttttaatgttggatttggtattatttcagcagatgcatcggagggagaaaacagaggaagtacgtttgcctattatggctcccagaatcttttaaaagagggagcaagttttcccttttatggaaaaaaccataatcgggttggcctttggccatatgggattaccaatgtagaaggcggccattcagcacctggatactgcctggaaccgaataagtccatgcgttcgggcactccgggaacgattgtaacatatgaccttgatacggatggtgacaacctgccccttgggctgacaagagaggatgcggaaattttatggtacgccctctcatcatcaggtaattttgaaggaggcatttcgggaaatgggaaaatcggccagggacattatattttggggcaggcagctacctgggccattatgtcaggaaattggaatgggttagatgatttccgtagtcagatggaagtcctaatcgagaacctgaaagatcctatgcttgcggtattgacaagaggagcattggaacaattttttaaacaggttaatggagccgtggaggaaggagccgttccaccctttgcatctaaatttcaaagccaggcgccggtacataaaatgaaagaaaacggggatgggacctatagtattacattggagtttgacggtgatgattggaggcagtccacgcttgtttatgatttgccagaagggtggaatgtgtctctggaacatggcagaatcacatttacatgtacgacaggtaatcccgatattggccttgtgaggggacattttcaggatggctccctgggggctcaatactgggttaagccaaatagctttaaaatctggtatccggatggttggaacgagagcagtgcagtagatgggaaacaggccatgattacaatggcagggaaacaggaatcgtgggaggtttggctgtcttttggaaaaagtacaacacaccgaggggaaggagactatgaaattccatatacccagtaccttcatgaggagacctttaagcgggattatgtcattgaactggaaaagcagtgttccgagacagggaaaactttggaaaactccacctttgaagtattagaaaagtttgagttttcgcagcttgacgggacaaatctggaaaaagaccagttcatgaaaatggtgcccacatcagaagggaaatttgaagatttaactgtgtgcgatatggggcttggtacagatgccaacggacatttttcgcattcggataagaagctttataaatatgagaagacttattgtgggggacacccagatccagtcattcattatgtggatggtgactctgactcggccgatgaagaaaatgaacgccggaagaagaaagcctgggatgcctggcaggagtgcgtggactggtgtgaggaaaattgtgatttccattccatcgatgagggcgttgccagagacttaatggaggaggaccgggatgaggcatggaatacctatattcacctgaagcgtatttacacagtcagggaaacagacgccagaaccggatatatccttcatgacctgcacaatgatgatgtctccattgaaattgttgaattttcatcttctcagtcagaaggagagggtgctattaccggctattaccctggcaatcgggcagtttcggttcgagagatggaggatgtgccgcagttgtcaaaatcggagaaggttacagatattgttcaggcgggaatgtcagatgaagggaatcaaaccaatgattcagttaccggacaggaaaaaatacctgaggataaagggaatgaacaagagcctgctactaaggaacatgagaacacaaagaaacatgaatcggaatcttccggtgacacagcagagaacggcgaggaaggggagaaggaggtggaggccgggtcagaggaaacagcagatacaacagaaataaaagagtctgatgaggcatcccagccatcaatggaaaacagagaggaaacagatgaagaggccagtcctgaagctgaaacgcaggattctggaaatgaagctgaatcagtggaagaagagaccataacagatgaccttcctaccgtagcaaccagatcccagatagggatagaaaaggacgtggccacaccgtcaaatgcgtccacctatttggtttcacgcccggcctctataaaagttggaacaggcgggcactgggaatgggatggtgtccaggaagacagtgaagtggcaccgattgagcaaggcagctatccgtccgggtacattggttacgcttatctggtgaaggatcacaggacagaaggagagctgcatattaataagcgggataaagaactttttgagctggaagaggacagctatggaaaagcccaagcagatgcaaccttagagggcgctgtatatggactatatgcggcagaggacatcaggcatccagatgggaaaaccggtgttgtattttctgcgggggagttggtatccattgccacaacggataaaaatggtgatgcttcattcttaaccataacagaagtatcagaaacctcaagagaggtaccgaatctctatacgggtaatgaagtgcggaatggaaatggctggattggccgtccgcttattttaggcagttattatatagaagaaatctcacgctctgaaggttatgaacgttctgtaaccggcaagaacttatctgagagcaatcgaacaggaaagcctattgtcttaaccgcgtcagggagtgcctatacagatggatttacccatagtattaatgaatggtttgaggattcctatgattttacggttaaatattataaaacaaagggtttcgatattctgctttccggtctcccggagcgtgtcaaggcatatgaggtgacaagaaaagagactgcatcacaggagcaggtgataaccggaacagagtgcgtagagaaaaaagatgcgcagggaaatatcctgtaccggactgccgagggtggagaatacaagttagacgaaaccgggaataagattataaaatcagatgatagcggcaatccggtgttgtctgaccgggcgctcacccaaacggtttcggcagtgaaccgattaaatgattatatctcatccattgagcgggaggaaccgtccgatttggatatggaagaatcagaagatattgacgaggattatattttgtgggagacgtcatcggctcttgctctttcggggtataagagtgggctgtctgattacccgtttaaaaaactcaaattatctggaaagacaaatgggaagataatagatgagattctgaccttctgctcctcggaaagtttttgggatgcatatgatgtagaggcagtatatgaggaggcgggaacctggtatgcaagaattcgttatggatataaagcattggcgaaccagccagccatttatgaaagcggcagtggtcttttagtaatccgaaaggaatatgagggcgggttttattatgcagtctatgaagagggacagtatgatatggatggttaccgcttcacagtagagaaaaaggaactggacctggaggctttggggcaagacgaaattctgttaaagacggtttatgcacctgtctatgagacctatgcgactggggagttcatattggacagtgaaggacagaaaatcccgcagatggaggcggttccaatctataccagccaggaggtggtttcctatgaggaaattctgacccctgtaaagacaaccgcagtggagggccaggtgagcatccatgtggatacggacgaggaatttacagagggggaaaaacatgagagaacttaccgggtagtgacagaacagaaaataacagaatatacagcaaccgtgaatgtcatgacgaccaaacctgcacagaagagtggctcttatctaaagttccctgtattattctatccgggccagtatgaaatctatgaagataacggcacacggaaagagccggttattatgctggagcgggtgattaagcaggcaatcgaggtgaaaaaggatattgctctggactcctatgagcataatacctatgagattcaccgtgacccgtttaccgttctttttggagggtataacggaacacaggagacgaagacgcttcccggctttttctttaaactgtatctgcgttctgatttagagaaaacagacaagcttatgaaaaaggaagatggcagttatgactatgtaacatttttcaaggaaaatcccgatgctgcgtctgagcttgcaattgagtgggatttggaaaagtatgatgcagacggggatatgacaaccgtacatgcaaaccggggcggcgggaaggacgactactggggacaaagcaggatgcttccttacggtacatatgttttggtggagcagcagccaacaggcatcccacaaaaacattatgagattgatgcgcctcaggaggtagagattccttttgttccacagatagatgccgatggtacagtacatgataaaatcccatctaaggaatatctttacgactctgccatgacaccggaggaattgactgagcgttatcagattcgatttaatgaagaaacccatatcatatatgcgcataataatgacggcgattttgaagtgtttaagtatggtctggagccggacagcagaagggattgccagaatgagacagtagcgaggtattatcattacggatctatcagcgaggatgctggaagtgcagaccaggtgtattatgaaacttattacgaccgggatggaacgatagcggattatggtgtaacaatgaatggagtggttaccatgaccggaaagtctactgccgtggaccggatgtacgcgaaagcacttgtaccctggagtgtgttagatcccagatatggagaagtaatcaacgatgacggcgatatcggaaaccgggaagctggcctggagacagatggaacctttaattttatttcctttgcagtcaaggattttgagaatgagttttacagctccagactcaggattgagaaattggattctgaaactggagaaaatattctgcatgaaggagcactctttaagatttatgcagccaaaagggacgtgattggaaatggtgcttccggtgtgacaggaagcggggatatcctgtttaatgaggagggaacacctctctatgaggaacgtgaacagattttcatgcaggacgacaccggagcggaagtaggggtttttaaggcttacaccacaatccgggacggagaagttgaaggagaggacggaagcctgcatacagagaagcagtgcgtggggtatctggaaacgtatcagccgttaggcgccggcgcttatgttctggtggaggtagaggcaccggatggatatgtgaagtcgaaacctattgcatttacggtatacagcgacaaggtggaatattatgaagacggcaatcaggagaaaaggacgcaggcggtaaaataccagtacatgcgtcccattggcgctgacgggaaaaccgttgtagaggatatgcaccagattattgtaaagaatgcacctacacatatagaaatacataagctggaaaaccgtgcggatgccgtcacataccgtgtagagggagatgaaaagcagctaaataaccgcggggacgtggatttacagtataaacccaatggggaatttgccgggtttggttatgtgacaaaacgccttgaaaacggtcaggagaagatatatgtggagaatgcaacactgacattgtatgaggggttggaagtaaagcagactggggaacacgagtatgagaaagtcaaggtgaagagaaatctatttggctcagtaacgggaatccaggcgtatgataccggcgtggataccgatataagacagactgggacaaacgcagcagggcaggcggagtgggatattacagaagaggataatccacccgtggacatatggtattttgacttaaaatatgatcccacagaacttgatgaacagaccggcatcctctatggactggatgactgggggaaccgtctctgtatgttggattcagaaacaggtatggcttatgtgacagataaaaccggcgcagtgattgtctggccgcttgatgaaaatggggataaaatcatttcccaatccgtagaagtatataccaacggggaaggaaattcatctatcaacatggatttgcagccagtaccagatgaaaacgggcttcctatctattataaggatggcggtgttatatggattgagaatgaatgggtgacggaccatggtgcctgtgaaattgcaagagtaaggcagggcgcctatatcctggaggagacagctgccccgttgtcagacggttatgtacagtccgcatcagtcggtgtgattgtccgtgatgtaagcgagaagcagtcctatgtgatggaggatgattacacaaagattgaggtatcaaagcttgatatgacttccagaaaagagattgagggcgcggttttaacactctacgaagcataccgcgtctatgattattccgtccgggggtggcatttggaaattctccgggatatggaggacaaaccgattgtagcggaacgatgggtgtcagaaggaaacgttccacactggattgaccatatcatgccgggagattatattcttcaggaaacaagagtaccaacaaaggccggctatgtgacggcggaggatgtggaagtcaccatattggaaaccagtgaagtacaaggctatgtaatggaagatgatcatacagcggtagaggttttaaagctggactcccggaccggggctgtgatggataatcttcaccgggccacactggctttatacgaagcccaggtagatgagaatggggaagtgcagtatagagatgatggaaccatactctatcatcaggaaaagaaggtgtatgaatggcagacagatgatggaagtgatgtgagaaaaacggcccatcaggttaccataccgggtggacacagctacacggcctatgattatgagatagagcaggttccgggaaccagtcaggcggtttgctatatcacggagacaggagccatgcgttttgagtatcttccggtggggaaatatgtgttggtagaagagcaggctccttacggctatacagtggcagcaccggtttatgtaccagtccttgatgtgggttctaaggaacgtgtgcagaccattaccatgacagacgaaccgattcaggtactcctgacaaaagtgaatgtgacaggtggaaaagaaatttccggagccacagttgccgtctaccgtgcaaaggaagatggtacacttgccaaacaccaaatgaaagacaaaaacgggaatctgttgtatgtaaccgatacggacgggaatctgttgcaggatgaagacgggaaccatattccggctatggagtacgaagaggcgtatctggtggaacgctggatttccggctcagacggaacatacacgaagcgggaccagaaagaaggcaggataccggaaggttatgaaatcggggatttaaggcttcatgagttaagtcagccagccgcagggaattactattttgttgaggaacagtctccctttggatatgtgagggctgcagaactgccgtttgcgattgttgatacactggatattcaaaagattgaactggtaaatgaactgattctgggccaggttgagattatcaagaccgataaaaggaatccggaacaagttttatcaggtgcaaggttccgattgtccaatctggataccaatgtggcgaccattctgataacggattccgatggtcgggcagtcagctccccggtccccattggagggataggaacggatggggcggtaagcctctatcatttccggatacaggaggtggaagcgcctgacggttatctgcttgacccgacagtccacgacttccagtttaatataaagacagaccagtaccagacgctcacctatcagtatgaagcagcggatagccctaatagggttatcatttctaaaaagcagcttacaacaaaggaggaactgccgggcgccagcttagaggtccgcagagtcacagagatggttgacaaggacggaacggttatacggattgacggtgatgtgattgaaagctggatttccacggaggtcccgcacgaactggaatgcctgacagagggaatgtatgtcctaatcgagacacgggcaccggaaggatatatagaagcagagaaagtgtactttacaatatccgggaacatgacagtggatgacatgcctatggtggagatgtttgatgatgacaccaagattgaaatcagaaaggtggacagtgaaaacggaaatcctttaaagggtgcgaagatgcagctgattttggaagcgtccggggaaatcattaaggaatggattacagatgaaaccggagcgattcagttcttcggtctcccggcaggcgtgtacttagtgaaagaggtagaggctccggaaggataccagataccggaaggtcccatgcggattaccgtgaccaaagattataagcaacagacgtttgtaatggagaaccggatgaccgaattgatgattgacaaactggacgaggagaccagggaaccggtaacaggagcagtgctgcagcttacagatggggaaggaaatcaggtggcgaaatgggttaccactggggaaccggaactaatccgcggcttaaaggcaggctggtatatactggaggaaatccaggcggcagacggctatctgcttttaaaggaaccagtgagaatcgagattacggagaaagcaggaatacaaaccgttaccattacaaaccggaaacttgaagtggaagtggcaaaaacagataaagagaccggagaaaacctggcgggagcaagacttcagttgattcgagatgccgatggcatggtattgaaagaatgggtaagcgggaagatacctgaaatttttaaaggactttctgtcggagaatatacaatcagggaattgacggccccagagggctatgcagtaacagggcgtttaaacttttgcgtgagcggaaccgaggaaaagcaggagattaacctggtaaatgagaagattgtggtggaactccagaagacggacaccttggaaggcagcccagtggaaggggcggttcttcagcttctaaaggctgcggggacaaacgaggagactgtaatgaaagaatgggtgtcaggaaaggagccgcttattttaacaggaataccgtctggtatctatacaataagggaaacacaggcaccggaaggatatgtcccaatggaggatatgaaaattgagatccttcctaatcagaccatgcagcattttgatattaaaaaccagccaattcagatagagataggaaaggttagcggagaaacaggaaaactgctgggcggcgctgttcttcaactggtaagagattcggacggtacagtaatccggaaatggacatccagggacggtgaggctgaacactttaaaaaccttgcgggcggtacgtatataatacgtgaggtgaaggctccctccgggtatgagaaaatggagccgcaggaaatagaaataaaagatattgaggcgatacaggaatttgtggtcaagaactataaaatcacccattcaggtggcggcggtggcagcactccgaatcacccgagaccctcagcggagtatatggaactgtttaagattgatggaagaactggccagaagcttgcaggggcaaagtttaccgtttacaatccagacggcagtgtctatgcggaaggatttacaaatgcagcaggaacattccggtttaagaaacctttaaaaggagcctatacatttaaagagacggaggcaccagagggatactatctaaatgaggtactgcatgcgtttgcagtaactgaagatgggaccatagaaggggatacgactatggaaaactacagtaaaacagaaatgataatctctaaagtggatgtaacaacagcagaggaactgcttggcgccgagatcgaggtcacagaccaggacggaaaccatatcttctccggcattagtgatgaaaagggaaaagtatattttccggtcccgtctccgggggaatatcatttcagggaactgacagcaccggaagggtatgacagaaatgagacggtcttttcattcacggtgtttgaggacggcagtataattggcgacaataccattaccgatcagaaacattacggaacaatcacggccaattatgagacaaatagaaggggagagggtgatttgacggtcggtgaacttaatcatgcaccaaagacaggagataccagctattttgtaggggtgttcatggcatggcttgcatccgtagtaagcctgttaattgtttccctctcaaagtggaggaaaaaacaaaagaaatcaaaaacagggataaaggctggcatgtttctattattggcaataatggtagttagtacgccggtgatggagtctcaggcagcggaagatgtaattgaaaatatatatgaggaacaccagtatacaacggaaaaccccgattctaatgaagcagaaaagctgtttgaaaaagaaattgagagagacggaaaaaaataccgtctctcagaaatccggacagaagtgctggaagaacaggggaaaaggagttctggaaattcttttgaaatcgcaacaagcccgtttattgatggaaaagtggaggttaggccgaaagaacagataacccggaatggaattacttatcatttggtaaaatcccagaaggaagcagccgttatacgggctcatgaggtaccggtaagtgaggaagtcttatatgaagcagtggaagccaaggacatgataccgtcaaaaatcaagacgacggtaacagatgagaaaaccgggcagatgatggagactattgcaaaaatttctgaccagacatttggcggggagcgcctggacgatacattttccttttcagctgtgttccatgaatatgggctggacggctattggattggggaccaggtttttaaactgagtgggactgaaccagacttcaccggatatgaagggcagctcctttctctgattgaggctgatgctgagcattataccatagaagcagcaaggtgggatggggaagcttacacagatgaagcaggtatcctatgccgtagggttgttataactggtaccaagaaggtgagtgactgcacagtgatgtatgagggaagtgcctattttccggaggaggaaggcgtacgcctgatttctacctatgatagcggggaagaagaggtagagcaatacaccatgaaagcaacgggggtttacatacctaaaaagaaccatggagcagcggtcgccactgtgattggtgttaccagtgtcggtgcaggtactgcaggatatacctattatcggaaaaagaagcagaatcaaaacgtataa

SEQ ID NO: 15 - DNA, 699 bp, Clostridium hathewayi
  DSM 13479
atgaaaaggatactatccagtgccatacaagtatttaagcaaatcaaaagcgaccctatgatgtttgcggcttgcttcaccccttttattatgggagctttaatcaaatttggtattccattttttgaaagaataacaaagttttctttacaaggatactatccgatttttgatttattgctttctatcatggctcctgtactgctttgttttgcatttgccatgattacgttagaggaaattgatgataaagtatcgcggtacttttcaattacccctcttggtaaggcggggtatctctttacaaggttggtagtacccgcaattatttcagcggtcattgcttttatcgtacttttgcttttctcattagaaaagctacccactagaatgatgattggtttagcacttctcggttcggtacaggcaatcattgtttcacttatgattattaccttatccggtaataaattagagggtatggctgtaacaaaactttctgcacttacactgttaggcattccagttcctttctttatagatagttactaccagttcgcggttggcttcctcccatcattttgggtagcaaaagccgtacagaatgaagcagttctttattttcccacagcattggtagtagctttgatctggtactactttcttataaaacgtctgtttcggaagctggcaggataa

SEQ ID NO: 16 - DNA, 237 bp, Roseburia intestinalis
ttgcagaaaaaagaagatggaatcattttgaaaaaaatattaattgctttaattaagttttatagaaaatatctttctccgatgaaaacgaccaagtgtccatattgcccgacctgttctttatatgggttggaggcagttgaaaagtacggagctctaaaagggggagcacttgctttatggagaatcttaagatgtaatcctttttcaaaaggtggatatgatccagttccatag

SEQ ID NO: 17 - DNA, 549 bp, Unknown
  Description of Unknown Bacterial polynucleotide
atggttactgtaaataattctgactattataacaacattccagctggcagggatttaaacaagctacccaataattccagcgccggtacaactgtaggatatcagtatgatggacttacagaggaagatcgtttcgtacaaaaagttttgcgggagcattatgataaaatgtacaaagaaaatatgtcgcattctgatccaatggcatatgtcatatcgaaatattgtgatgtgacttcacctaacttttgctcatatatgacagaagaccaacgttccatagcttaccgtacagagaaaagaatgttacaatccggaggaaaacctgtgggtggatttgcacggtatgattatgcattaaggaattacaaggatgtatatacaggtggttcaagaagtgttggttatgtacgcaatactgacagggaaaaacagcatgccagaagtgttgtaaatcagcaaatttcaaatctactttcagagaatgggatatcaatatcaaaacaggcaatttggtattttctattgacccatatacatatcaactaa

SEQ ID NO: 18 - DNA, 600 bp, Clostridium sp.
  L2-50
atgtttattacctgtttggacctggaaggagtattagtaccggaaatctggatcgcattcgctgaggccagcggcatcccggagttaaagcgcaccacccgcgatgagccggactatgacaagctgatgaaatggcgtctcggaattttaaaggagcacggacttggcttgaaggagatccaggaaaccatcgagaagatcgacccgatgccgggagcaagagcgttcttagatgagctccgggagctgggacaggtaatcatcatcagcgataccttcacccagttcgcaaagccactcatgaaaaagctgggctggccaaccattttctgcaacgagctggaagtagcagaggatggtgagatcaccggattcagaatgcgcattgagcagtccaagctcagtaccgtcaaggcactgcagtccatcggctttgagaccattgccagcggcgacagctacaatgaccttggcatgatccgcgccagcaaggccggcttcctctttaagagcaccgatcagatcaagaacgacaatccggatcttccggcgtacgagacctatgaggagctgctggcagcgatcaaggcagcagtataa

SEQ ID NO: 19 - DNA, 315 bp, Unknown
  Description of Unknown Bacterial polynucleotide
atgatatccacagtcacaaaagcccatgcacagcaggaaatcctgctcctcgccatgaaggccgggcagatccagctggaaaacggagcggaaatctttcgcgtggaagataccatcatgcatatctgccgcgcctacggacttcattctgtccatattttcgtgctgagtaacggtatttttctaagctgcggagatgagacggaacccctgtttgccaaggttttgcaggtgcctgtcaacaataccaacctgcgcagagttgcggaagtaaaccagctatcaaggcgcattgaggaagaagggctttccccg

SEQ ID NO: 20 - DNA, 654 bp, Clostridium sp.
tgggctgttgcgaccgcgcatcggaagaatgcttacgaaagcaagatcggaaccgcggaagaaaaagccagggaaataatagatgaagcgttaaagacggcagagacaaagaagcgagaagctctcctggaggcgaaggaagagtccttaaagactaagaatgagctggataaagagacaaaggaaagaagagctgaacttcagcgctatgaacgacgtgtgctgagcaaagaagaaaacttagacaaaaaaacagagaaccttgaacggcgggaagccgggcttgcatcccgtgaggaagccttgaacaagcgtaatggtgaggttgaggccctttacgaaaaagggatacaggaactggagcgtatttccggtttaacctccgaacaggcaaaagagtatctgctcagatctgttgaggcggaggtcaagcatgacactgccaagatgatcaaggatctggagaacaaggcaaaagaagaagctgacaaaaaggcaaaggagtatgtggttactgcgattcagagatgtgctgcagaccatgtggctgaaactaccgtatctgtagtacagcttccgaacgatgaaatgaagggacgcatcattggccgtgagggacgtaacatccgtacccttgagactatgactggtgtg

(19) United States
(12) Patent Application Publication
(10) Pub. No.: US 2015/0376697 A1
(43) Pub. Date: Dec. 31, 2015

(54) METHOD AND SYSTEM TO DETERMINE BIOMARKERS RELATED TO ABNORMAL CONDITION

(71) Applicants: Shenghui Li, Shenzhen (CN); Junjie Qin, Shenzhen (CN); Jianfeng Zhu, Shenzhen (CN); Dongya Zhang, Shenzhen (CN); Zhuye Jie, Shenzhen (CN); Jun Wang, Shenzhen (CN); Jian Wang, Shenzhen (CN); Huanming Yang, Shenzhen (CN)

(72) Inventors: Shenghui Li, Shenzhen (CN); Junjie Qin, Shenzhen (CN); Jianfeng Zhu, Shenzhen (CN); Dongya Zhang, Shenzhen (CN); Zhuye Jie, Shenzhen (CN); Jun Wang, Shenzhen (CN); Jian Wang, Shenzhen (CN); Huanming Yang, Shenzhen (CN)

(73) Assignees: BGI-Shenzhen, Shenzhen (CN); BGI Shenzhen, Co., Limited, Shenzhen (CN)

(21) Appl. No.: 13/640,448

(22) PCT Filed: Aug. 22, 2012
(86) PCT No.: PCT/CN12/80479

§ 371 Date: May 18, 2015

(30) Foreign Application Priority Data
Aug. 1, 2012 (CN) PCT/CN2012/079524

(51) Int. Cl. C12Q 1/68

(52) U.S. Cl. CPC C12Q 1/6883; C12Q 2600/112

(57) ABSTRACT

A method and system to determine biomarkers related to abnormal condition in a subject are provided, comprising:sequencing nucleic acid samples from a first and a second subject in order to obtain multiple sequences respectively consisting of the first and the second sequencing results, wherein the first subject is in the abnormal condition; and the second subject is not in the abnormal condition; and the nucleic acid samples from the first and the second subject are both isolated from the samples of the same type; and the first and the second subject belong to the same species; and determining the biomarkers related to the abnormal condition in the subject based on the difference between the first and the second sequencing results.

### CROSS-REFERENCE TO RELATED APPLICATION

The present patent application claims benefits of and priority to PCT Patent Application No. PCT/CN2012/079524, filed Aug. 1, 2012, which is incorporated herein by reference.

### TECHNOLOGY FIELD

The present invention relates to the field of biotechnology, and in particular, to a method and system to determine the biomarkers related to abnormal condition.

### BACKGROUND

Metagenomics is also known as environmental genomics, yuan genomics, ecological genomics, or community genomics. It is a subject of directly studying the microbial communities in natural state, including the total genome of cultured and uncultured bacteria, fungi and viruses. In 1998, Handelsman et al, from the department of plant pathology of University of Wisconsin, firstly proposed the concept of metagenomics in the study of soil microbes. The conventional microbial study is restricted by isolation and pure culture technology of microorganism. Whereas metagenomics study is based on the microbial community in the specific environment, with research purposes of microbial diversity, population structure, evolutionary relationships, functional activity, collaborative relationships with each other and environmental relationship between the new microorganisms. The metagenomics basic research strategy includes environmental genomic fragments of DNA extraction and purification, library construction, target gene screening and/or large-scale sequencing analysis. Metagenomic library contains both the cultured and uncultured microbial genes and genomes. Clone DNA in a natural environment to the host cell culture, thus avoiding the problem of isolating and culturing microorganisms. In this study, by means of large-scale sequence analysis combined with bioinformatics tools, a lot of unknown microbial genes or new gene cluster can be found on the basis of gene sequence analysis. It is of great significance for understanding the microbial flora composition, evolutionary history and metabolic characteristics, and mining new genes with potential applications. However, the current research of metagenomic still needs to be improved.

### SUMMARY

Embodiments of the present disclosure seek to solve at least one of the problems existing in the prior art. The present invention provides a method and system to determine biomarkers related to an abnormal condition in a subject.

According to the embodiments of a first broad aspect of the present disclosure, a method is provided to determine biomarkers related to an abnormal condition in a subject, comprising: sequencing nucleic acid samples from the first and the second subject in order to obtain multiple sequences respectively consisting of the first and the second sequencing results, wherein the first subject is in the abnormal condition; and the second subject is not in the abnormal condition; and the nucleic acid samples from the first and the second subject are both isolated from the samples of the same type; and the first and the second subject belong to the same species; and determining the biomarkers related to the abnormal condition in the subject based on the difference between the first and the second sequencing results.

According to the embodiments of present disclosure, the method to determine biomarkers related to an abnormal condition in a subject may further possess the following additional features:

According to one embodiment of present disclosure, the abnormal condition is a disease.

According to one embodiment of present disclosure, the disease is selected from at least one of neoplastic diseases, autoimmune diseases, genetic diseases and metabolic diseases.

According to one embodiment of present disclosure, the abnormal condition is diabetes.

According to one embodiment of present disclosure, the first and the second subject are human.

According to one embodiment of present disclosure, the nucleic acid samples from the first and the second subject are isolated from excreta of the first and the second subject respectively.

According to one embodiment of present disclosure, sequencing nucleic acid samples from the first and the second subject is conducted by means of second-generation sequencing technology or third-generation sequencing technology.

According to one embodiment of present disclosure, the sequencing step is conducted by means of at least one apparatus selected from Hiseq 2000, SOLID, 454, and True Single Molecule Sequencing.

According to one embodiment of present disclosure, determining the biomarkers related to the abnormal condition based on the difference between the first and the second sequencing results further comprises: aligning the first and the second sequencing results against reference gene catalogue; and determining relative abundance of gene respectively in the nucleic acid samples from the first and the second subject based on the alignment result; and conducting statistical tests on the relative abundance of each genes in the nucleic acid samples from the first and the second subject; and determining gene markers which are significantly different between the nucleic acid samples from the first and the second subject based on their relative abundances.

According to one embodiment of present disclosure, before aligning the first and the second sequencing results against reference gene catalogue, a filtering step is used to remove contamination sequence. The contamination sequence is at least one sequence from adapter sequence, low quality sequence, and host genome sequence.

According to one embodiment of present disclosure, the step of aligning is conducted by means of at least one of SOAP 2 and MAQ, which aligns the first and the second sequencing results against reference gene catalogue, optionally the human gut microbial flora non-redundant gene catalogue.

According to one embodiment of present disclosure, the method further comprises: performing de novo assembly and metagenomic gene prediction on high quality reads from the first and the second sequencing results, wherein the genes do not match with the reference gene catalogue are defined as new genes; and integrating the new genes with the human non-redundant gene catalogue to obtain an updated gene catalogue; and conducting taxonomic assignment and functional annotation.

According to one embodiment of present disclosure, taxonomic assignment is performed by aligning every gene of reference gene catalogue against IMG database.

According to one embodiment of present disclosure, aligning every gene of reference gene catalogue against IMG database is conducted by BLASTP method to determine taxonomic assignment of the gene, using the 85% identity as the threshold for genus assignment and another threshold of 80% of the alignment coverage. For each gene, the highest scoring hit(s) above these two thresholds was chosen for the genus assignment. For the taxonomic assignment at the phylum level, the 65% identity was used instead.

According to one embodiment of present disclosure, functional annotation is performed by aligning putative amino acid sequences, which have been translated from reference gene catalogue, against the proteins/domains in eggNOG or KEGG database.

According to one embodiment of present disclosure, aligning putative amino acid sequences, which have been translated from reference gene catalogue, against the proteins/domains in eggNOG or KEGG database is conducted by BLASTP method to determine functional annotation of the gene, according to functions whose E-Value less than 1e-5.

According to one embodiment of present disclosure, the relative abundances comprise species and functions relative abundances, and reference gene catalogue comprises taxonomic assignment and functional annotation. Determining the biomarkers related to the abnormal condition based on the difference between the first and the second sequencing results further includes: aligning the first and the second sequencing results against a reference gene catalogue; and determining species and functions relative abundances of each genes respectively in the nucleic acid samples from the first and the second subject based on the alignment result; and conducting statistical tests on the species and functions relative abundances of each genes in the nucleic acid samples from the first and the second subject; and determining species and functions markers respectively which are significantly different between the nucleic acid samples from the first and the second subject based on their relative abundances. Optionally, after obtaining the relative abundances, the Poisson distribution is used to conduct the statistical test on accuracy of the relative abundances.

According to one embodiment of present disclosure, the method further comprises enterotypes identification.

According to one embodiment of present disclosure, the method further comprises assessing the effect of each covariate, optionally, enterotype, T2D, age, gender and BMI. Preferably, Permutational Multivariate Analysis Of Variance method is used.

According to one embodiment of present disclosure, the method further comprises correcting population stratifications of the data, wherein adjust the gene relative profile, preferably, by using EIGENSTRAT method in order to remove the covariate effect.

According to one embodiment of present disclosure, the statistical test is conducted by at least one of Student T test and Wilcox rank sum test.

According to one embodiment of present disclosure, the method further comprises clustering the gene markers and advanced assembling to construct organisms genome associated with the abnormal condition.

According to one embodiment of present disclosure, the method further comprises steps to validate the biomarkers.

According to one embodiment of a second broad aspect of present disclosure, a system is provided to determine biomarkers related to abnormal condition in a subject, comprising: a sequencing apparatus, which is adapted to sequence nucleic acid samples from the first and the second subject in order to obtain multiple sequences respectively consisting of the first and the second sequencing results, wherein the first subject is in the abnormal condition, the second subject is not in the abnormal condition, the nucleic acid samples from the first and the second subject are both isolated from the samples of the same type, the first and the second subject belong to the same species; and an analytical apparatus, which is connected to the sequencing apparatus, and adapted to determine the biomarkers of the abnormal condition in the subject based on the difference between the first and the second sequencing results.

According to the embodiments of present disclosure, the system to determine biomarkers related to abnormal condition in a subject may further possess the following additional features.

According to one embodiment of present disclosure, the system further comprises a nucleic acid sample isolation apparatus, which is connected to the sequencing apparatus, and adapted to isolate nucleic acid sample from the subjects, optionally from their excreta.

According to one embodiment of present disclosure, the sequencing apparatus is adapted to carry out second-generation sequencing platform or third-generation sequencing platform.

According to one embodiment of present disclosure, the sequencing apparatus is adapted to carry out at least one apparatus selected from Hiseq 2000, SOLID, 454, and True Single Molecule Sequencing.

According to one embodiment of present disclosure, the analytical apparatus further comprises:

means for alignment, which is adapted to align the first and the second sequencing results against a reference gene catalogue; and

means for determining relative abundance, which is connected to the means for alignment and adapted to determine relative abundance of gene respectively in the nucleic acid samples from the first and the second subject based on the alignment result; and

means for conducting statistical tests, which is connected to the means for determining relative abundance and adapted to conduct statistical tests on the relative abundance of gene in the nucleic acid samples from the first and the second subject; and

means for determining markers, which is connected to the means for conducting statistical tests and adapted to determine gene markers which are significantly different between the nucleic acid samples from the first and the second subject based on their relative abundances.

According to one embodiment of present disclosure, the analytical apparatus further comprises: means for filtering, which is connected to the means for alignment and adapted to a step of filtering to remove contamination sequence before aligning the first and the second sequencing results against the reference gene catalogue. The contamination sequence is at least one sequence from adapter sequence, low quality sequence, and host genome sequence.

According to one embodiment of present disclosure, the means for alignment is at least one of SOAP 2 and MAQ, which aligns the first and the second sequencing results against reference gene catalogue, optionally, against the human gut microbial flora non-redundant gene catalogue.

According to one embodiment of present disclosure, the relative abundances comprise species and functions relative abundances, and reference gene catalogue comprises taxonomic assignment and functional annotation. The system further comprises:

means for determining relative abundance, which is adapted to determine species and functions relative abundances of gene respectively in the nucleic acid samples from the first and the second subject based on the alignment result; and

means for conducting statistical tests, which is adapted to conduct statistical tests on the species and functions relative abundances of gene in the nucleic acid samples from the first and the second subject; and

means for determining markers, which is adapted to determine species and functions markers which are significantly different between the nucleic acid samples from the first and the second subject based on their relative abundances.

According to one embodiment of present disclosure, the means for conducting statistical tests is conducted by at least one of Student T test and Wilcox rank sum test.

According to one embodiment of present disclosure, the system further comprises a genome assembling apparatus, which is adapted to cluster the gene markers and advanced assemble to construct organisms genome associated with the abnormal condition, preferably, by Identification of Metagenomic Linkage Group (MLG).

According to the embodiment of present disclosure, the method to determine biomarkers related to an abnormal condition in a subject (also known as MGWAS (a two-stage case-control Metagenome-Wide Association Study)) based on the high throughput sequencing technologies can conduct association study between metagenomics and disease to discover biomarkers related to the disease. As for the highly improved throughput sequencing technologies and the significantly reduced cost, large population study can be implemented. Taking full advantage of reference gene catalogue can make the association analysis more reproducible and reliable. Meanwhile, through using multiple relevance statistical test method, the false positive caused by gene relative abundance estimation inflation is greatly reduced. The method can directly discover the biomarkers associated with target phenotype and the association analysis is of high reliability and accuracy.

Additional aspects and advantages of the embodiments of present disclosure will be given in part in the following descriptions, which become apparent in part from the following descriptions, or be learned from the practice of the embodiments of the present disclosure.

### BRIEF DESCRIPTION OF DRAWINGS

These and other aspects and advantages of the present disclosure will become apparent and more readily appreciated from the following descriptions taken in conjunction with the drawings, in which:

FIG. 1 shows the flow diagram of the method to determine biomarkers related to an abnormal condition according to one embodiment of present disclosure.

FIG. 2 shows the flow diagram of the method to determine biomarkers related to an abnormal condition according to another embodiment of present disclosure.

FIG. 3 shows the flow diagram of the system to determine biomarkers related to an abnormal condition according to one embodiment of present disclosure.

FIGS. 4 to 6 show the flow diagram of the method to determine biomarkers related to an abnormal condition according to embodiments 3, 4, and 5 of present disclosure.

FIG. 7, according to one embodiment of present disclosure, shows detection error rate distribution of relative abundance profiles in different sequencing amount. The X axis represents the sequencing amount of a sample, which was defined as the number of paired-end reads, and the Y axis represents the relative abundance of a gene. The 99% confidence interval (CI) of the relative abundance was estimated and the detection error rate was defined as the ratio of the interval width to the relative abundance itself. The scaled detection error rate, transformed by log₁₀(log₁₀(1+x)), was used to color all the points, with darker color representing larger detection error rate. Two indifference curves were added: detection error rate that fall to the upper right of the curves would be less than 1× and 10×, respectively.

### DETAILED DESCRIPTION

In the following detailed description of the embodiments of present disclosure, the embodiment examples are shown in the drawings, wherein the same or a similar label to the same or similar elements or components of the same or similar functions. The following embodiments described by reference drawings are exemplary, which is only used to explain the present invention, and not regarded as the limitations of the present invention.

It should be noted, the term “first” and “second” is only used for describing, and can not be regarded as implying the relative importance or indicating the number technical features specified in the instructions. As a result, characteristics limited by “first”, “second” can express or imply one or more of the characteristics. Further, in the description of the present invention, unless otherwise noted, “a plurality of” means two or more.

### A Method to Determine Biomarkers Related to an Abnormal Condition

According to the embodiments of a first broad aspect of the present disclosure, a method is provided to determine biomarkers related to an abnormal condition in a subject.

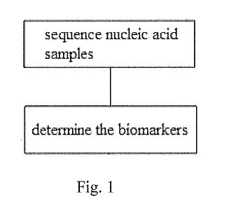

Referring to FIG. 1, the method to determine biomarkers related to an abnormal condition in a subject comprising following steps:

First, sequence nucleic acid samples from the first and the second subject in order to obtain multiple sequences respectively consisting of the first and the second sequencing results. According to one embodiment of present disclosure, the first and the second subject are in different conditions. Specifically, the first subject is in the abnormal condition and the second subject is not in the abnormal condition. And the nucleic acid samples from the first and the second subject are both isolated from the samples of the same type, with the first and the second subject belonging to the same species.

Next, after obtaining the first and the second sequencing results, determine the biomarkers of the abnormal condition in the subject based on the difference between the first and the second sequencing results. Because the first and second subject are from the same species and their nucleic acid samples are from the same type, the difference between the first and the second sequencing results can reflect the biomarkers associated with the abnormal condition.

Used herein, the term “abnormal condition” should have a broad understanding of the subjects (organisms) referring to any condition different from the normal condition, including both physiological abnormalities and psychological abnormalities. According to the embodiment of the present disclosure, the type of the disease used by the present invention is not subject to special restrictions. According to one embodiment of present disclosure, the disease is selected from at least one of neoplastic diseases, autoimmune diseases, genetic diseases and metabolic diseases. According to a specific embodiment of the present disclosure, the abnormal condition is diabetes. As a result, it is effective to discover biomarkers of specific species and specific disease by using the method of the present invention. According to the embodiment of the present disclosure, the range of the term “subject” is not limited and can be any organisms. According to one embodiment of present disclosure, the first subject and the second subject are human. Thus, according to the embodiment of the present disclosure, the first subject can be a patient with a specific disease, and the second subject can be a healthy person. In addition, according to the embodiment of the present disclosure, the number of the first and the second subject is not limited, and it can be a plurality of subjects. In this way, it can make the biomarkers determined more reliable.

According to the embodiment of present disclosure, the source of nucleic acid samples is not limited on the condition that they are from the same type sources. According to one embodiment of present disclosure, the nucleic acid samples from the first and the second subject are isolated from excreta of the first and the second subject respectively. In this way, it can effectively identify gut microbiome information and effectively discover the relationship between gut microbiome and specific disease.

According to the embodiment of present disclosure, the sequencing technologies are not limited. According to one embodiment of present disclosure, sequencing nucleic acid samples from the first and the second subject is conducted by means of second-generation sequencing technology or third-generation sequencing technology. According to a specific embodiment of present disclosure, the sequencing step is conducted by means of at least one apparatus selected from Hiseq 2000, SOLID, 454, and True Single Molecule Sequencing. In this way, it can take advantage of features of high throughput and depth in sequencing from the sequencing apparatus, which provide benefits to the following data analysis, specially, statistical test in precision and accuracy.

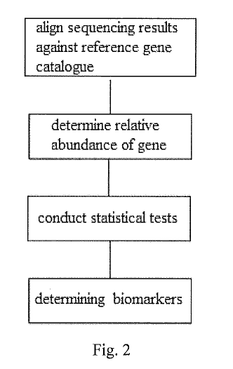

According to the embodiment of present disclosure, the inventors can conduct any methods to analysis the sequence results. According to one embodiment of present disclosure, referring to FIG. 2, determining biomarkers is by following steps:

First, align the first and the second sequencing results against a reference gene catalogue. According to the embodiment of present disclosure, the reference gene catalogue is not limited and can be newly constructed or any known database, for example, the human gut microbial flora non-redundant gene catalogue. According to one embodiment of present disclosure, before aligning the first and the second sequencing results against a reference gene catalogue, a step of filtering is used to remove contamination sequence. According to the embodiment of present disclosure, the contamination sequence is at least one sequence from adapter sequence, low quality sequence, and host genome sequence. In this way, it helps to improve efficiency of alignment and then improve efficiency of biomarkers determined. According to the embodiment of present disclosure, the tool used to align the first and the second sequencing results against the reference gene catalogue can be any known means. According to one embodiment of present disclosure, the step of aligning is conducted by means of at least one of SOAP 2 and MAQ, which aligns the first and the second sequencing results against a reference gene catalogue. In this way, it helps to improve efficiency of alignment and then improve efficiency of biomarkers determined.

Next, determine relative abundance of gene respectively in the nucleic acid samples from the first and the second subject based on the alignment result. By aligning the sequencing reads against the reference gene catalogue, built up a corresponding relationship between sequencing reads and genes of the reference gene catalogue. So that corresponding sequence reads relative number can reflect the gene relative abundance effectively, aiming at specific gene of nucleic acid samples. Thus, through alignment result and statistic analysis, determine the relative gene abundance in the nucleic acid samples. According to the embodiment of present disclosure, optionally, after obtaining the relative abundances, preferably, the Poisson distribution is used to conduct the statistical test on the accuracy of the relative abundances. Specifically, the inventors used the method developed by Audic and Claverie (1997) to assess the theoretical accuracy of the relative abundance estimates. Given that the inventors have observed xᵢ reads from gene i, as it occupied only a small part of total reads in a sample, the distribution of xᵢ is approximated well by a Poisson distribution. Let us denote N the total reads number in a sample, so N=Σᵢxᵢ. Suppose all genes are the same length, so the relative abundance value αᵢ of gene i simply is αᵢ=xᵢ/N. Then the inventors could estimate the expected probability of observing yᵢ reads from the same gene i, is given by the formula below,

$$P ( {a}_{i}^{′} \vert {a}_{i} ) = P ( {y}_{i} \vert {x}_{i} ) = \frac{( {x}_{i} + {y}_{i} ) !}{{x}_{i} ! {y}_{i} ! {2}^{( {x}_{i} + {y}_{i} + 1 )}}$$

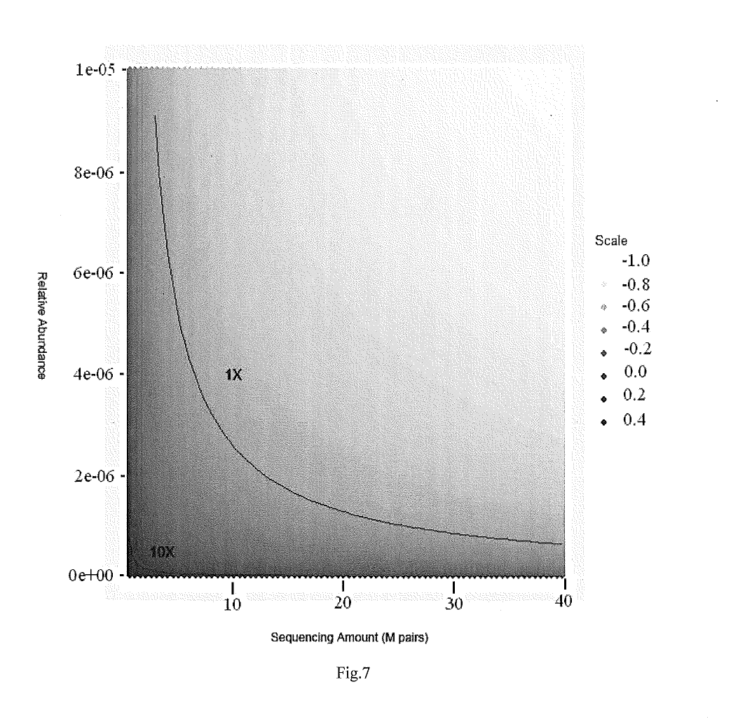

Here, αᵢ′=yᵢ/N is the relative abundance computed by yᵢ reads. Based on this formula, the inventors then made a simulation by setting the value of αᵢ from 0.0 to 1e-5 and N from 0 to 40 million, in order to compute the 99% confidence interval for αᵢ′ and to further estimate the detection error rate (shown in FIG. 7).

Finally, after determining relative abundance of gene in the nucleic acid samples, conduct statistical tests on the relative abundance of gene in the nucleic acid samples from the first and the second subject in order to determine gene markers which are significantly different between the nucleic acid samples from the first and the second subject based on their relative abundances. If existing the gene significantly different, the gene is regarded as a biomarker related to the abnormal condition, namely gene marker.

According to the embodiment of present disclosure, the term “biomarker” should have a broad understanding, that is any detectable biological indicators reflecting the abnormal condition, which comprises gene marker, species marker (species/genus marker) and functions marker (KO/OG marker).

In addition, according to the embodiment of present disclosure, the method further comprises: performing de novo assembly and metagenomic gene prediction on high quality reads from the first and the second sequencing results, wherein the genes, if not matched with the reference gene catalogue, are defined as new genes; and integrating the new genes with the reference gene catalogue to obtain an updated gene catalogue. Thus, the capacity of reference gene catalogue is enlarged in order to promote efficiency of biomarkers determined. According to one embodiment of present disclosure, taxonomic assignment is performed by aligning every genes of a reference gene catalogue against IMG database. According to one embodiment of present disclosure, aligning every genes of a reference gene catalogue against IMG database is conducted by BLASTP method to determine taxonomic assignment of the gene, using the 85% identity as the threshold for genus assignment and another threshold of 80% of the alignment coverage. For each gene, the highest scoring hit(s) above these two thresholds was chosen for the genus assignment. For the taxonomic assignment at the phylum level, the 65% identity was used instead. Thus, taxonomic assignment of the gene can be determined effectively. According to one embodiment of present disclosure, functional annotation is performed by aligning putative amino acid sequences, which have been translated from a reference gene catalogue, against the proteins/domains in eggNOG or KEGG database. According to one embodiment of present disclosure, aligning putative amino acid sequences, which have been translated from reference gene catalogue, against the proteins/domains in eggNOG or KEGG database is conducted by BLASTP method to determine functional annotation of the gene, according to functions whose E-Value less than 1e-5. Thus, functional annotation of the gene can be determined effectively.

In addition, as for the known or newly constructed reference gene catalogue, the taxonomic assignment and functional annotation of a gene may be included. In this way, based on the gene relative abundances, perform taxonomic assignment and functional annotation of the gene, and then determine species and functions relative abundances. Further, determine species and functions markers related to an abnormal condition. Thus, according to one embodiment of present disclosure, the relative abundances comprise species and functions relative abundances, and reference gene catalogue comprises taxonomic assignment and functional annotation. Determining the biomarkers related to the abnormal condition based on the difference between the first and the second sequencing results further includes: aligning the first and the second sequencing results against reference gene catalogue; and determining species and functions relative abundances of gene respectively in the nucleic acid samples from the first and the second subject based on the alignment result; and conducting statistical tests on the species and functions relative abundances of gene in the nucleic acid samples from the first and the second subject; and determining species and functions markers respectively which are significantly different between the nucleic acid samples from the first and the second subject based on their relative abundances. Optionally, after obtaining the relative abundances, preferably, the Poisson distribution is used to conduct the statistical test on accuracy of the relative abundances. The method to determine species and functions relative abundances is not limited. According to the embodiment of present disclosure, conduct statistical tests on the gene relative abundances from the same species and from the same functional annotation respectively, for example, summation, average, median values and so on, to determine species and functions relative abundances. According to one embodiment of present disclosure, the formula to calculate gene relative abundances is as follow.

For any sample S, the inventors calculated the abundance as follows:
Step 1: Calculation of the copy number of each gene:

$${b}_{i} = \frac{{x}_{i}}{{L}_{i}}$$

Step 2: Calculation of the relative abundance of gene i

$${a}_{i} = \frac{{b}_{i}}{{\sum }_{j} {b}_{j}} = \frac{\frac{{x}_{i}}{{L}_{i}}}{{\sum }_{j} \frac{{x}_{j}}{{L}_{j}}}$$

αᵢ: The relative abundance of gene i in sample S.
Lᵢ: The length of gene i.
xᵢ: The times which gene i can be detected in sample S (the number of mapped reads).
bᵢ: The copy number of gene i in the sequenced data from sample S.

According to one embodiment of present disclosure, the statistical test of gene, species and functions relative abundances is not limited. According to one embodiment of present disclosure, the statistical test is conducted by at least one of Student T test and Wilcox rank sum test.

The gut microbiome of human in normal condition can be divided into three enterotypes, which are not correlated with other covariate like age, gender and so on and also not affected by chronic metabolic diseases like obesity. Hence, estimate each sample enterotype and perform population stratification analysis to remove the enterotype effect on usual disease-gut microbial flora association analysis are needed because some true markers may be uncovered due to enterotype. The relative abundances of a genus was estimated and used for identifying enterotypes from Chinese samples. The inventors used the same identification method as described in the original paper of enterotypes. In the study, samples were clustered using Jensen-Shannon distance. In fact, the inventors can also use other cluster methods like Hierarchical clustering algorithm. And the enterotype result can be validated through functions relative abundances. On other aspect, association test may be affected by covariate like enterotype, T2D, age, gender and BMI. Such effect can also be removed by population stratification analysis. Use Permutational Multivariate Analysis Of Variance method to assess the effect of each covariate and correct population stratifications of the data, wherein adjust the gene relative profile preferably by using EIGENSTRAT method in order to remove the covariate effect.

After obtaining gene markers, according to one embodiment of present disclosure, the method further comprises clustering the gene markers and advanced assembling to construct organisms genome associated with the abnormal condition, preferably, by Identification of Metagenomic Linkage Group (MLG). For obtained gene markers, in general, many of them are possibly from the related low-amount species and many species in human gut are uncultured and not isolated successfully. A method to cluster the genes is used and after that the inventors rebuilt its genome to get more microbiome information which related to disease. The known cluster algorithm can also be applied to cluster genes. After clustering, the inventors selected the paired-end reads from gene markers by alignment method like SOAP2. De novo assembly like SOAPdenovo was performed on the selected reads to construct microbial genome. Furthermore, modifying and improvement will be made on genome by applying composition-based binning method. And this modifying procedure is repeated until that there are no further distinct improvements of the assembly, obtaining microbial draft genome.

According to one embodiment of present disclosure, the method further comprises steps to validate the biomarkers. Thus, the efficiency and reliability of association between biomarkers and abnormal condition, optionally, disease like diabetes, are improved.

### A System to Determine Biomarkers Related to Abnormal Condition

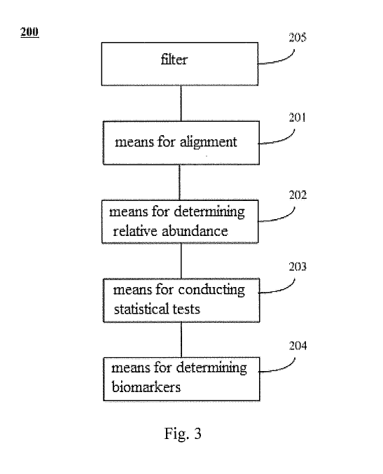

According to one embodiment of a second broad aspect of present disclosure, a system is provided to determine biomarkers related to an abnormal condition in a subject. The system 1000, referring to FIG. 3, comprises sequencing apparatus 100 and analytical apparatus 200. According to one embodiment of present disclosure, sequencing apparatus 100 which adapted to sequence nucleic acid samples from the first and the second subject in order to obtain multiple sequencing sequence respectively consisting of the first and the second sequencing results, wherein the first subject is in the abnormal condition; and the second subject is not in the abnormal condition; and the nucleic acid samples from the first and the second subject are both isolated from the samples of the same type; and the first and the second subject belong to the same species. According to the embodiment of present disclosure, analytical apparatus 200, which is connected to sequencing apparatus 100 and adapted to determine the biomarkers of the abnormal condition in the subject based on the difference between the first and the second sequencing results. In this way, using the system 1000 can determine biomarkers related to abnormal condition, according to the embodiment of present disclosure, and then biomarkers related to abnormal condition can be determined effectively.

According to one embodiment of present disclosure, the system 1000 further comprises nucleic acid sample isolation apparatus 300, which is connected to the sequencing apparatus 100 and adapted to isolate nucleic acid sample from the subjects, optionally from their excreta. Thus, the nucleic acid sample isolation apparatus 300 provides the sequencing apparatus 100 nucleic acid samples to sequence. According to the embodiment of present disclosure, the method and equipment used to sequence are not limited. According to the embodiment of present disclosure, the sequencing apparatus 100 is adapted to carry out second-generation sequencing platform or third-generation sequencing platform. According to one embodiment of present disclosure, the sequencing apparatus 100 is adapted to carry out at least one apparatus selected from Hiseq 2000, SOLID, 454, and True Single Molecule Sequencing. Combining with newest sequencing technology and aiming at single site which can reach higher sequence depth, detection sensitivity and accuracy are greatly promoted. In this way, it can take advantage of features of high throughput and depth in sequencing from the sequencing apparatus, which improves detection analysis on the nucleic acid samples and benefits the following data analysis, specially, statistical tests in precision and accuracy.

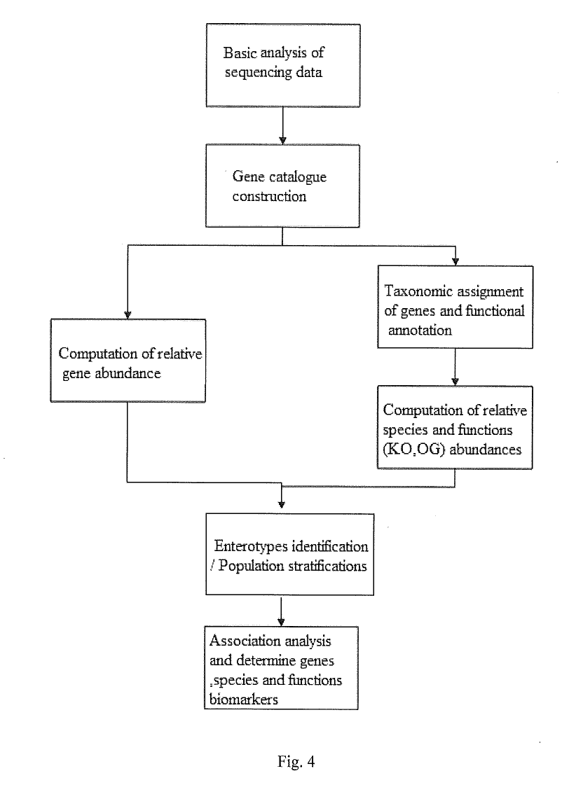

Referring to FIG. 4, according to one embodiment of present disclosure, the analytical apparatus 200 further comprises means 201 for alignment, means 202 for determining relative abundance, means 203 for conducting statistical tests and means 204 for determining markers. According to one embodiment of present disclosure, means 201 for alignment is adapted to align the first and the second sequencing results against reference gene catalogue. Means 202 for determining relative abundance is connected to means 201 for alignment and adapted to determine relative abundance of gene respectively in the nucleic acid samples from the first and the second subject based on the alignment result. Means 203 for conducting statistical tests is connected to means 202 for determining relative abundance and adapted to conduct statistical tests on the relative abundance of gene in the nucleic acid samples from the first and the second subject. Means 204 for determining markers is connected to means 203 for conducting statistical tests and adapted to determine gene markers which are significantly different between the nucleic acid samples from the first and the second subject based on their relative abundances. According to one embodiment of present disclosure, the means 203 for conducting statistical tests is conducted by at least one of Student T test and Wilcox rank sum test.

According to one embodiment of present disclosure, the analytical apparatus 200 further comprises: means 205 for filtering, which is connected to means 201 for alignment and adapted to extract high quality reads by filtering low quality reads in order to remove contamination sequence before aligning the first and the second sequencing results against reference gene catalogue. The contamination sequence is at least one sequence from adapter sequence, low quality sequence, host genome sequence. According to one embodiment of present disclosure, the means 201 for alignment is at least one of SOAP 2 and MAQ, which aligns the first and the second sequencing results against reference gene catalogue. The reference gene catalogue can be stored at means 201 for alignment, optionally, the human gut microbial flora non-redundant gene catalogue is stored. Thus the efficiency of alignment is promoted.

According to one embodiment of present disclosure, the relative abundances comprise species and functions relative abundances, and reference gene catalogue comprises taxonomic assignment and functional annotation. The system further comprises: the means for determining relative abundance, which is adapted to determine species and functions relative abundances of gene respectively in the nucleic acid samples from the first and the second subject based on the alignment result; and the means for conducting statistical tests, which is adapted to conduct statistical tests on the species and functions relative abundances of gene in the nucleic acid samples from the first and the second subject; and the means for determining markers, which is adapted to determine species and functions markers which are significantly different between the nucleic acid samples from the first and the second subject based on their relative abundances. Thus, species and functions markers related to abnormal condition are determined effectively.

With the system 1000 to determine biomarkers related to abnormal condition according to the embodiment of present disclosure, the method to determine biomarkers related to an abnormal condition according to the embodiment of present disclosure can be implemented effectively. As for the advantages of this method, the above has been described in detail. It should be noted, skilled in the art can understand the same. The above described features and advantages of the method to determine biomarkers related to abnormal condition are also suitable for the system to determine biomarkers related to an abnormal condition. For the convenience of description, they are not repeated here.

### DETAILED DESCRIPTION

The present invention is further exemplified in the following non-limiting examples.

Unless otherwise stated, the technical means used in the examples are well-known conventional to the skilled in the art, referring to “Laboratory Manual For Molecular Cloning” (third edition) or related products, and the reagents and products are all commercially available. Not stated in detail, the various processes and methods are conventional to the public in this field, and the source of the reagents, trade names and its composition needed to set out are indicated when it first appears. Unless otherwise stated, the same reagents used subsequently are in accordance with the first indicated instructions.

### Example 1

### Sample Collection

All 344 fecal samples from 344 Chinese individuals living in the south of China were collected by Shenzhen Hospital of Peking University. The patients who were diagnosed with type 2 diabetes (T2D) Mellitus according to the 1999 WHO criteria (Alberti, K. G. & Zimmet, P. Z. Definition, diagnosis and classification of diabetes mellitus and its complications. Part 1: diagnosis and classification of diabetes mellitus provisional report of a WHO consultation. Diabetic medicine: Journal of the British Diabetic Association 15, 539-553, doi:10.1002/(SICI)1096-9136(199807)15:7<539::AID-DIA668>3.0.CO;2-S (1998), incorporated herein by reference) constitute the case group in the study, and the rest non-diabetic individuals were taken as the control group (shown in Table 2). Patients and healthy controls were asked to provide a frozen fecal sample. Volunteers pay attention to 3 days' diet before sampling, and eat light, but not high fat foods. And in the 5 days before sampling, volunteers didn't eat yogurt and other lactic acid products and prebiotics. The samples were collected not to mix with urine, and isolated from human pollution and air.

TABLE 2
Sample collection

Samples

| Sample | T2D | Obesity | Stage I | Stage II |
|---|---|---|---|---|
| DO | Yes | Yes | 32 | 73 |
| DL | Yes | No | 39 | 26 |
| NO | No | Yes | 37 | 62 |
| NL | No | No | 37 | 38 |

### Example 2

### DNA Extraction and Sequencing

2.1 Fecal Samples Storage

Fresh fecal samples were taken into the sterilized stool collection tube, and samples were immediately frozen by storing in a home freezer. Frozen samples were transferred to the place to store, and then stored at −80° C. until analysis.

2.2 DNA Extraction

Frozen aliquot (200 mg) of each fecal sample was suspended in 250 μl of guanidine thiocyanate, 0.1 M Tris (pH 7.5) and 40 μl of 10% N-lauroyl sarcosine. DNA was extracted as previously described (Manichanh, C. et al. Reduced diversity of fecal microbiota in Crohn's disease revealed by a metagenomic approach. Gut 55, 205-211, doi: gut. 2005.073817 [pii]10.1136/gut.2005.073817 (2006), incorporated herein by reference). DNA concentration and molecular size were estimated using a nanodrop instrument (Thermo Scientific) and agarose gel electrophoresis.

2.3 DNA Library Construction and Sequencing

DNA library construction was performed following the manufacturer's instruction (Illumina). The inventors used the same workflow as described elsewhere to perform cluster generation, template hybridization, isothermal amplification, linearization, blocking and denaturation, and hybridization of the sequencing primers.

The inventors constructed one paired-end (PE) library with insert size of 350 bp for each samples, followed by a high-throughput sequencing to obtain around 20 million PE reads. The reads length for each end is 75 bp-90 bp (75 bp and 90 bp read length in stage I samples; 90 bp read length for stage II samples).

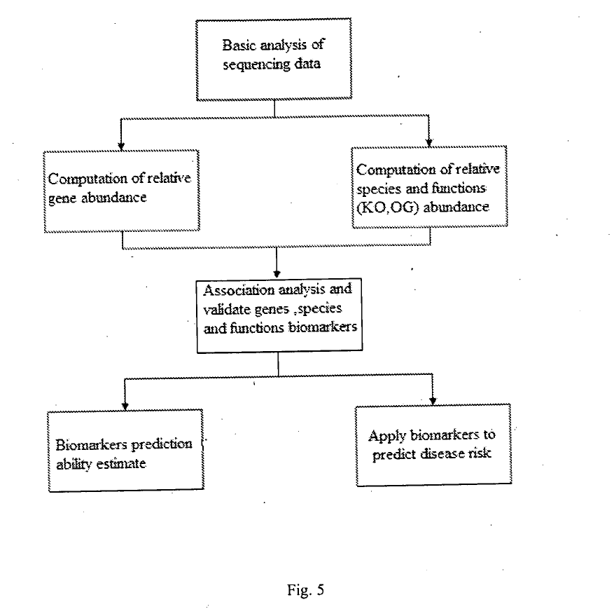

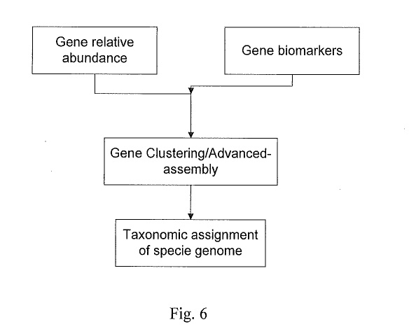

Referring to FIG. 4 to 6, the flow diagrams show the method to determine biomarkers related to T2D, comprising several main steps as follows:

### Example 3

### Identification of Biomarkers

3.1 Basic Analysis of Sequencing Data

After obtaining sequencing data from 145 samples of stage I, high quality reads were extracted by filtering low quality reads with ‘N’ base, adapter contamination or human DNA contamination from the Illumina raw data, totaling 378.4 Gb of high-quality data. On average, the proportion of high quality reads in all samples was about 98.1%, and the actual insert size of the PE library ranges from 313 bp to 381 bp.

3.2 Gene Catalogue Updating

Employing the same parameters that were used for building the MetaHIT gene catalogue (Junjie Qin, Ruiqiang Li, JeroenRaes, et al. (2010) A human gut microbial gene catalogue established by metagenomic sequencing. Nature, 464:59-65, incorporated herein by reference), the inventors performed de novo assembly and gene prediction for 145 samples in stage I using SOAPdenovo v1.06 and GeneMark v2.7, respectively. All predicted genes were aligned pairwise using BLAT and genes, of which over 90% of their length can be aligned to another one with more than 95% identity (no gaps allowed), were removed as redundancies, resulting in a non-redundant gene catalogue comprising of 2,088,328 genes. This gene catalogue from the Chinese samples was further combined with the previously constructed MetaHIT gene catalogue, by removing redundancies in the same manner. At last, the inventors obtained an updated gene catalogue with 4,267,985 predicted genes. 1,090,889 of these genes were uniquely assembled from the Chinese samples.

3.3 Taxonomic Assignment of Genes

Taxonomic assignment of the predicted genes was performed using an in-house pipeline. In the analysis, the inventors collected the reference microbial genomes from IMG database (v3.4), and then aligned all 4.2 million genes onto the reference genomes using BLASTP. Based on the comprehensive parameter exploration of sequence similarity across phylogenetic ranks by MetaHIT enterotype paper, the inventors used the 85% identity as the threshold for genus assignment (Arumugam, M. et al. Enterotypes of the human gut microbiome. Nature 473, 174-180, doi:10.1038/nature09944 (2011), incorporated herein by reference), as well as another threshold of 80% of the alignment coverage. For each gene, the highest scoring hit(s) above these two thresholds was chosen for the genus assignment. For the taxonomic assignment at the phylum level, the 65% identity was used instead. Here, 21.3% of the genes in the updated catalogue could be robustly assigned to a genus, which covered 26.4-90.6% (61.2% on average) of the sequencing reads in the 145 samples; the remaining genes were likely to be from currently undefined microbial species.

3.4 Functional Annotation

The inventors aligned putative amino acid sequences, which had been translated from the updated gene catalogue, against the proteins/domains in eggNOG (v3.0) and KEGG databases (release 59.0) using BLASTP (e-value≦1e-5). Each protein was assigned to the KEGG orthologue group (KO) or eggNOG orthologue group (OG) by the highest scoring annotated hit(s) containing at least one HSP scoring over 60 bits. For the remaining genes without any annotation in eggNOG database, the inventors identified novel gene families based on clustering all-against-all BLASTP results using MCL with an inflation factor of 1.1 and a bit-score cutoff of 60. Using this approach, the inventors identified 7,042 novel gene families (≧20 proteins) from the updated gene catalogue.

3.5 Quantification of Metagenome Content

3.5.1 Computation of Relative Gene Abundance

The high quality reads from each sample were aligned against the gene catalogue by SOAP2 using the criterion of “identity>90%”. In the sequence-based profiling analysis, only two types of alignments could be accepted: i), an entirety of a paired-end read can be mapped onto a gene with the correct insert-size; and ii). one end of the paired-end read can be mapped onto the end of a gene, only if the other end of read was mapped outside the genic region. In both cases, the mapped read was counted as one copy.

Then, for any sample 5, the inventors calculated the abundance as follows:

Step 1: Calculation of the copy number of each gene:

$${b}_{i} = \frac{{x}_{i}}{{L}_{i}}$$

Step 2: Calculation of the relative abundance of gene i

$${a}_{i} = \frac{{b}_{i}}{{\sum }_{j} {b}_{j}} = \frac{\frac{{x}_{i}}{{L}_{i}}}{{\sum }_{j} \frac{{x}_{j}}{{L}_{j}}}$$

αᵢ: The relative abundance of gene in sample S.
Lᵢ: The length of gene i.
xᵢ: The times which gene i can be detected in sample S (the number of mapped reads).
bᵢ: The copy number of gene i in the sequenced data from sample S.

Based on gene relative profiles and the kwon taxonomic assignment and functional annotation of genes from above, one can sum up the gene relative abundances from the same species and from the same functional annotation respectively in order to obtain species relative abundance profiles and functions relative abundance profiles.

3.5.2 Estimation of Profiling Accuracy.

The inventors used the method developed by Audic and Claverie (Audic, S. & Claverie, J, M. The significance of digital gene expression profiles. Genome Res 7, 986-995 (1997), incorporated herein by reference) to assess the theoretical accuracy of the relative abundance estimates. Given that the inventors have observed xᵢ reads from gene i, as it occupied only a small part of total reads in a sample, the distribution of xᵢ is approximated well by a Poisson distribution. Let us denote N the total reads number in a sample, so N=Σᵢxᵢ. Suppose all genes are the same length, so the relative abundance value αᵢ of gene i simply is αᵢ=xᵢ/N. Then the inventors could estimate the expected probability of observing yᵢ reads from the same gene i, is given by the formula below,

$$P ( {a}_{i}^{′} \vert {a}_{i} ) = P ( {y}_{i} \vert {x}_{i} ) = \frac{( {x}_{i} + {y}_{i} ) !}{{x}_{i} ! {y}_{i} ! {2}^{( {x}_{i} + {y}_{i} + 1 )}}$$

Here, αᵢ′=yᵢ/N is the relative abundance computed by yᵢ reads. Based on this formula, the inventors then made a simulation by setting the value of αᵢ from 0.0 to 1e-5 and N from 0 to 40 million, in order to compute the 99% confidence interval for αᵢ′ and to further estimate the detection error rate (shown in FIG. 7).

3.5.3 Construction of Gene, KO, and OG Profile

The updated gene catalogue contains 4,267,985 non-redundant genes, which can be classified into 6,313 KOs (KEGG Orthologue) and 45,683 OGs (orthologue group in eggNOG, including 7,042 novel gene families). The inventors first removed genes, KOs or OGs that were present in less than 6 samples across all 145 samples in stage I. To reduce the dimensionality of the statistical analyses in MGWAS, in the construction of gene profile, the inventors identified highly correlated gene pairs and then subsequently clustered these genes using a straightforward hierarchical clustering algorithm. If the Pearson correlation coefficient between any two genes is >0.9, the inventors assigned an edge between these two genes. Then, the cluster A and B would not be clustered, if the total number of edges between A and B is smaller than |A|*|B|/3, where |A| and |B| are the sizes of A and B, respectively. Only the longest gene in a gene linkage group was selected to represent this group, yielding a total of 1,138,151 genes. These 1,138,151 genes and their associated measures of relative abundance in 145 stage I samples were used to establish the gene profile for the association study.

For the KO profile, the inventors utilized the gene annotation information of the original 4,267,985 genes and summed the relative abundance of genes from the same KO. This gross relative abundance was taken as the content of this KO in a sample to generate the KO profile of 145 samples. The OG profile was constructed using the same method used for KO profile.

3.6 Enterotypes Identification

The relative abundance of a genus was estimated by the same method used in construction of KO profile, and then was used for identifying enterotypes from the Chinese samples. The inventors used the same identification method as described in the original paper of enterotypes (Arumugam, M. et al. Enterotypes of the human gut microbiome. Nature 473, 174-180, doi:10.1038/nature09944 (2011), incorporated herein by reference). In the study, samples were clustered using Jensen-Shannon distance.

$$JSD ( P ‖ D ) = \frac{1}{2} D ( P ‖ M ) + \frac{1}{2} D ( Q ‖ M )$$

in which:

$$M = \frac{1}{2} ( P + Q )$$

$$D ( P ‖ M ) = {\sum }_{i} P ( i ) ln \frac{P ( i )}{M ( i )} \\ D ( Q ‖ M ) = {\sum }_{i} Q ( i ) ln \frac{Q ( i )}{M ( i )}$$

P (i) and Q (i) are the relative abundances of gene i in sample P, Q respectively. Enterotype of each sample can be validated by the same method on OG/KO relative profile.

3.7 Statistical Analysis of MGWAS

3.7.1 PERMANOVA

In the study, Permutational Multivariate Analysis Of Variance (PERMANOVA, McArdle, B. H. & Anderson, M. J. Fitting Multivariate Models to Community Data: A Comment on Distance-Based Redundancy Analysis. Ecology 82, 290-297 (2001), incorporated herein by reference) was used to assess the effect of each covariate including enterotype, T2D, age, gender and BM1, on four types of profiles. The inventors performed the analysis using the method implemented in R package—“vegan” (Zapala, M. A. & Schork, N. J. Multivariate regression analysis of distance matrices for testing associations between gene expression patterns and related variables. Proceedings of the National Academy of Sciences of the United States of America 103, 19430-19435, doi:10.1073/pnas.0609333103 (2006), incorporated herein by reference), and the permuted P-value was obtained by 10,000 times permutations.

| | | | P-values (top 20 |
|---|---|---|---|
| | No. | P-values (original | principal components |
| Variables | subjects | gene profile) | in original gene profile) |
| Enterotypes | 3 | 0.0001 | 0.0001 |
| T2D | 2 | 0.0305 | 0.0004 |
| BMI | 255 | 0.3308 | 0.1851 |
| Gender | 2 | 0.2129 | 0.1326 |
| Age | 63 | 0.2030 | 0.1044 |

3.7.2 Population Stratifications.

To correct population stratifications of the data, the inventors used a modified version of the EIGENSTRAT method (Price, A. L. et al. Principal components analysis corrects for stratification in genome-wide association studies. Nature genetics 38, 904-909, doi:10.1038/ng1847 (2006), incorporated herein by reference) allowing the use of covariance matrices estimated from abundance levels instead of genotypes. However, as much of the signal in the data might be driven by the combined effect of many genes and not by just a few genes as assumed in GWAS studies, the inventors modified the method further by replacing each PC axis with the residuals of this PC axis from a regression to T2D. The number of PC axes of EIGENSTAT was determined by Tracy-Widom test at a significance level of P<0.05.

3.7.3 Statistical Hypothesis Test on Profiles

In stage I, to identify the association between the metagenome profile and T2D, a two-tailed Wilcoxon rank-sum test was used in the profiles that were adjusted for non-T2D-realted population stratifications. Then, while examining the stage I markers in stage II, a one-tailed Wilcoxon rank-sum test was used instead. Because the T2D is the primary factor impacting on the profile of examined gene markers in stage II, the inventors didn't adjust the population stratification for these genes.

3.7.4 Estimating the False Discovery Rate (FDR) and the Power

Instead of a sequential P-value rejection method, the inventors applied the “qvalue” method proposed in a previous study (Storey, J. D. A direct approach to false discovery rates. Journal of the Royal Statistical Society—Series B: Statistical Methodology 64, 479-498 (2002), incorporated herein by reference) to estimate the false discovery rate (FDR). In the MGWAS, the statistical hypothesis tests were performed on a large number of features of the gene, KO, OG and genus profiles. Given that a FDR was obtained by the qvalue method, the inventors estimated the power Pₑ for a given p-value threshold by the formula below,

$${P}_{e} = \frac{{N}_{e} ( 1 - {FDR}_{e} )}{N ( 1 - {π}_{0} )}$$

Here, π₀ is the proportion of null distribution P-values among all tested hypotheses; Nₑ is the number of P-values that were less than the P-value threshold; N is the total number of all tested hypotheses; FDR_{d} is the estimated false discovery rate under the P-value threshold.

3.8 Selection of Biomarkers

In stage I the inventors use two-side Wilcox test based on population-adjusted stage I gene and functions (KO and OG) relative abundance profile and the inventors adjust the multiple test by estimating the false discovery rate (FDR). Finally the gene passing the test was the biomarkers. At last, the inventors use a clustering method to cluster the genes into species biomarkers (called MLG). And the inventors test the gene, functions (KO and OG), species biomarkers by Student T test. The p-value of each biomarkers are summarized in Table 2-1, Table 2-2 and Table 3.

To reduce and structurally organize the abundant metagenomic data and to enable us to make a taxonomic description, the inventors devised the generalized concept of Metagenomic Linkage Group (MLG) in lieu of a species concept for a metagenome. Here a MLG is defined as a group of genetic material in a metagenome that is likely physically linked as a unit rather than being independently distributed; this allowed us to avoid the need to completely determine the specific microbial species present in the metagenome, which is important given there are a large number of unknown organisms and that there is frequent lateral gene transfer (LGT) between bacteria. Using the gene profile, the inventors defined and identified a MLG as a group of genes that co-exists among different individual samples and has a consistent abundance level and taxonomic assignment.

3.9 Identification of Metagenomic Linkage Group (MLG)

3.9.1 the Clustering Method for Identifying MLG

In the present study, the inventors devised a concept of metagenomic linkage group (MLG), which could facilitate the taxonomic description of metagenomic data from whole-genome shotgun sequencing. To identify MLG from the set of T2D-associated gene markers, the inventors developed an in-house software that comprises three steps as indicated below:

Step 1: The original set of T2D-associated gene markers was taken as initial subclusters of genes. It should be noted that in the establishment of the gene profile the inventors had constructed gene linkage groups to reduce the dimensionality of the statistical analysis. Accordingly, all genes from a gene linkage group were considered as one subcluster.

Step 2: The inventors applied the Chameleon algorithm (Karypis, G. & Kumar, V. Chameleon: hierarchical clustering using dynamic modeling. Computer 32, 68-75 (1999), incorporated herein by reference) to combine the subclusters exhibiting a minimal similarity of 0.4 using dynamic modeling technology and basing selection on both interconnectivity and closeness. The similarity here is defined by the product of interconnectivity and closeness (the inventors used this definition in the whole analysis of MLG identification). The inventors term these clusters semi-clusters.

Step 3: To further merge the semi-clusters established in step 2, in this step, the inventors first updated the similarity between any two semi-clusters, and then performed a taxonomic assignment for each semi-cluster (see the method below). Finally, two or more semi-clusters would be merged into a MLG if they satisfied both of the following two requirements: a) the similarity values between the semi-clusters were >0.2; and b) all these semi-clusters were assigned from the same taxonomy lineage.

3.9.2 Taxonomic Assignment for a MLG

All genes from a MLG were aligned to the reference microbial genomes (IMG database, v3.4) at the nucleotide level (by BLASTN) and the NCBI-nr database (February 2012) at the protein level (by BLASTP). The alignment hits were filtered by both the e-value (<1×10-10 at the nucleotide level and <1×10-5 at the protein level) and the alignment coverage (>70% of a query sequence). From the alignments with the reference microbial genomes, the inventors obtained a list of well-mapped bacterial genomes for each MGL group and ordered these bacterial genomes according to the proportion of genes that could be mapped onto the bacterial genome, as well as the average identity of the alignments. The taxonomic assignment of a MLG was determined by the following principles: 1) if more than 90% of genes in this MLG can be mapped onto a reference genome with a threshold of 95% identity at the nucleotide level, the inventors considered this particular MLG to originate from this known bacterial species; 2) if more than 80% of genes in this MLG can be mapped onto a reference genome with a threshold of 85% identity at the both nucleotide and protein levels, the inventors considered this MLG to originate from the same genus of the matched bacterial species; 3) if the 16S sequences can be identified from the assembly result of a MLG, the inventors performed the phylogenetic analysis by RDP-classifier (bootstrap value>0.80) (Wang, Q., Garrity, G. M., Tiedje, J. M. & Cole, J. R. Naive Bayesian classifier for rapid assignment of rRNA sequences into the new bacterial taxonomy. Appl Environ Microbiol 73, 5261-5267, doi: AEM.00062-07 [pii]10.1128/AEM.00062-07 (2007), incorporated herein by reference) and then defined the taxonomic assignment for the MLG if the phylotype from 16S sequences was consistent with that from genes.

3.9.3 Advanced-Assembly for a MLG

To reconstruct the potential bacterial genomes, the inventors designed an additional process of advanced-assembly for each MLG, which was implemented in four steps.

Step 1: Taking the genes from a MLG as a seed, the inventors identified samples that contain the seed with the highest abundance among all samples, and then selected the paired-end reads from these samples that could be mapped onto the seed (including the paired-end read that only one end could be mapped). The lower limit of the coverage of these paired-end reads is 50× in no more than 5 samples, which is computed by dividing the total size of selected reads by the total length of the seed.

Step 2: A de novo assembly was performed on the selected reads in step 1 by using the SOAPdenovo with the same parameters used for the construction of the gene catalogue.

Step 3: To identify and remove the mis-assembled contigs probably caused by contaminated reads, the inventors applied a composition-based binning method. Contigs whose GC content value and sequencing depth value were distinct from the other contigs of the assembly result were removed, as they might be wrongly assembled due to various reasons.

Step 4: Taking the final assembly result from step 3 as a seed, the inventors repeated the procedure from step 2 until that there were no further distinct improvements of the assembly (in detail, the increment of total contig size was less than 5%).

3.10 MLG-Based Analysis

3.10.1 Validation of MLG Methods

The performance of the MLG identification methods was evaluated by following steps: 1) In the quantified gene result, the rarely present genes (present in <6 samples) were filtered at first; 2) Based on the taxonomic assignment result in the updated gene catalogue, the inventors identified a set of gut bacterial species by the criteria of containing 1,000˜5,000 unique mapped genes, with the similarity threshold of 95%. In this step, the inventors manually removed the redundant strains in one species and also discarded the genes that could be mapped onto more than one species. Ultimately, 130,065 genes from 50 gut bacterial species were identified as a test set for validating the MLG method; 3). The standard MLG method described above was performed on the test set. For each MLG, the inventors computed the percentage of genes that were not from the major species as an error rate (namely % gene, shown in Table 7).

3.10.2 Relative Abundance of a MLG

The inventors estimated the relative abundance of a MLG in all samples by using the relative abundance values of genes from this MLG. For this MLG, the inventors first discarded genes that were among the 5% with the highest and lowest relative abundance, respectively, and then fitted a Poisson distribution to the rest. The estimated mean of the Poisson distribution was interpreted as the relative abundance of this MLG. At last, the profile of MLGs among all samples was obtained for the following analyses.

### Example 4

### A Two-Stage Validation

4.1 Data Analysis

The inventors repeat Example 1 and Example 2 steps to get sequenced data and repeat Example 3 steps to get gene, functions and species relative profile with the use of 199 samples in stage II.

4.2 Validation of Biomarkers

In stage I the inventors use two-side Wilcox test based on population-adjusted stage I gene and functions (KO and OG) relative abundance profile and In stage II the inventors use one-side Wilcox test based on origin gene and functions (KO and OG) relative abundance profile and the side is determined by stage I genes direction. And the inventors adjust the multiple test by estimating the false discovery rate (FDR). Finally the gene passing the test was the biomarkers. At last, the inventors use a clustering method to cluster the genes into species biomarkers (called MLG). And the inventors test the gene, functions (KO and OG), species biomarkers by Student T test. The p-value of each biomarkers are summarized in Table 2-1, Table 2-2 and Table 3.

The inventors next control for the false discovery rate (FDR) in the stage II analysis, and define a total of 52,484 T2D-associated gene markers from these genes corresponding to a FDR of 2.5% (Stage II P value<0.01). The inventors apply the same two-stage analysis using the KO and OG profiles and identified a total of 1,345 KO markers (Stage II P<0.05 and 4.5% FDR) and 5,612 OG markers (Stage II P<0.05 and 6.6% FDR) that are associated with T2D.

TABLE 2-1
Gene markers

| gene | | | | se- | Taxonomy |
|---|---|---|---|---|---|
| markers | Enrichment^{b} | P-values | P-values | quence | assignment |
| ID ^{a} | (direction) | (stage I) | (stage II) | ID | (level) |
| 52049 | 0 | 1.59E−06 | 5.19E−06 | 1 | Unclassified |
| 66281 | 0 | 3.88E−06 | 1.80E−06 | 2 | Unclassified |
| 86279 | 0 | 6.31E−09 | 4.80E−05 | 3 | Unclassified |
| 337304 | 1 | 6.18E−07 | 0.000186 | 4 | Unclassified |
| 1224005 | 1 | 8.67E−11 | 2.91E−07 | 5 | Clostridium |
| | | | | | hathewayi |
| | | | | | DSM 13479 |
| 1238449 | 0 | 7.27E−07 | 3.43E−06 | 6 | Unclassified |
| 2005309 | 1 | 2.14E−05 | 2.01E−05 | 7 | Unclassified |
| 2060779 | 0 | 7.58E−07 | 1.93E−05 | 8 | Clostridiales |
| | | | | | sp. SS3/4 |
| 2370529 | 1 | 1.03E−07 | 7.37E−05 | 9 | Unclassified |
| 2581190 | 1 | 8.52E−06 | 5.30E−05 | 10 | Unclassified |
| 2746171 | 1 | 6.15E−11 | 8.60E−07 | 11 | Clostridium |
| | | | | | hathewayi |
| | | | | | DSM 13479 |
| 3182475 | 1 | 2.75E−07 | 0.000296 | 12 | Unclassified |
| 3247820 | 1 | 3.65E−09 | 2.09E−07 | 13 | Clostridium |
| | | | | | hathewayi |
| | | | | | DSM 13479 |
| 3250057 | 1 | 1.03E−09 | 1.74E−06 | 14 | Unclassified |
| 3253773 | 1 | 4.53E−09 | 1.56E−06 | 15 | Clostridium |
| | | | | | hathewayi |
| | | | | | DSM 13479 |
| 3646621 | 0 | 2.26E−06 | 9.52E−05 | 16 | Roseburia |
| | | | | | intestinalis |
| 3793132 | 0 | 6.58E−06 | 1.12E−06 | 17 | Unclassified |
| 3815768 | 0 | 2.69E−06 | 1.40E−05 | 18 | Clostridium |
| | | | | | sp. L2-50 |
| 4097912 | 0 | 1.32E−05 | 1.57E−06 | 19 | Unclassified |
| 4136092 | 0 | 1.16E−06 | 1.63E−06 | 20 | Clostridium |

^{a} Sequences of gene markers are shown in Table 9
^{b}1 represents T2D group enrichment and bad marker; 0 represents control group enrichment and good marker.

TABLE 2-2
Functions markers

| functions | Enrichment^{c} | P-values^{d} | P-values |
|---|---|---|---|
| markers | (direction) | (stage I) | (stage II) |
| COG0229 | 1 | 1.82E−19 | 1.26E−05 |
| K01251 | 1 | 6.10E−20 | 3.91E−05 |
| K00162 | 1 | 3.87E−10 | 2.06E−05 |
| K05396 | 1 | 3.92E−11 | 6.33E−06 |
| K07315 | 1 | 2.97E−12 | 1.45E−07 |
| COG0499 | 1 | 2.61E−18 | 0.000168 |
| NOG134456 | 1 | 2.62E−14 | 0.000137 |
| COG0659 | 0 | 7.88E−20 | 3.62E−08 |
| K03321 | 0 | 1.09E−22 | 6.22E−08 |
| K14652 | 0 | 7.60E−20 | 2.92E−07 |
| NOG303876 | 0 | 1.50E−16 | 2.86E−05 |
| K05339 | 0 | 1.42E−17 | 3.49E−07 |
| COG1283 | 0 | 1.72E−15 | 5.34E−06 |
| COG1266 | 0 | 1.70E−11 | 6.77E−06 |
| K03324 | 0 | 8.79E−20 | 1.51E−05 |

^{c}1 represents T2D group enrichment and bad marker; 0 represents control group enrichment and good marker.
^{d}The null hypothesis is that T2D groups don't differ from Control groups on the functions markers, P value (P value < 0.05, considering as significant) means the probability of obtaining a test statistic at least as extreme as the one that was actually observed, assuming that the null hypothesis is true.

TABLE 3
Species makers

| | Enrichment^{f} | P-values | P-values |
|---|---|---|---|
| MLG^{e}ID | (direction) | (stage I) | (stage II) |
| T2D-154 | 1 | 0.001347368 | 0.000254046 |
| T2D-140 | 1 | 0.000397275 | 0.002849677 |
| T2D-139 | 1 | 0.001328967 | 0.000211459 |
| T2D-11 | 1 | 4.16065E−08 | 7.58308E−05 |
| T2D-5 | 1 | 4.21047E−05 | 1.97056E−06 |
| T2D-80 | 1 | 0.000129893 | 1.40862E−05 |
| T2D-57 | 1 | 4.00759E−07 | 2.20525E−05 |
| T2D-15 | 1 | 4.74327E−05 | 0.00029675 |
| T2D-1 | 1 | 0.000601047 | 0.003604634 |
| T2D-7 | 1 | 0.000601047 | 0.000279527 |
| T2D-137 | 1 | 6.70507E−07 | 0.001204531 |
| T2D-165 | 1 | 0.009634384 | 0.00166131 |
| T2D-12 | 1 | 4.51685E−06 | 8.04282E−08 |
| T2D-8 | 1 | 7.08451E−10 | 9.94749E−06 |
| T2D-93 | 1 | 0.000208898 | 0.002040004 |
| T2D-62 | 1 | 7.62983E−06 | 0.000688358 |
| T2D-2 | 1 | 3.14293E−05 | 0.001850999 |
| T2D-6 | 1 | 0.000202468 | 0.002073171 |
| T2D-9 | 1 | 3.03578E−05 | 0.000117763 |
| T2D-14 | 1 | 4.16065E−08 | 7.44243E−07 |
| T2D-16 | 1 | 7.44638E−09 | 2.21532E−06 |
| T2D-30 | 1 | 0.000140727 | 0.004548142 |
| T2D-37 | 1 | 0.008582927 | 7.65392E−05 |
| T2D-73 | 1 | 2.54217E−06 | 0.002296161 |
| T2D-79 | 1 | 0.000511522 | 0.001924895 |
| T2D-90 | 1 | 0.000704982 | 0.001710744 |
| T2D-170 | 1 | 0.000665393 | 0.000421786 |
| Con-107 | 0 | 1.12113E−07 | 0.001826862 |
| Con-112 | 0 | 0.006389079 | 0.00019943 |
| Con-129 | 0 | 0.003274757 | 0.001001054 |
| Con-166 | 0 | 3.79947E−05 | 0.000193721 |
| Con-121 | 0 | 6.10793E−05 | 4.89846E−06 |
| Con-113 | 0 | 0.000284629 | 0.000972347 |
| Con-120 | 0 | 0.000190164 | 0.000540535 |
| Con-130 | 0 | 0.013361656 | 0.001837279 |
| Con-131 | 0 | 0.000898899 | 0.001737676 |
| Con-133 | 0 | 3.42674E−05 | 0.001474928 |
| Con-109 | 0 | 0.013510306 | 0.000167496 |
| Con-101 | 0 | 0.000136295 | 2.7876E−05 |
| Con-104 | 0 | 9.0896E−07 | 4.32913E−05 |
| Con-122 | 0 | 0.000415525 | 0.001694336 |
| Con-142 | 0 | 1.14239E−05 | 0.001163884 |
| Con-144 | 0 | 0.003671368 | 0.001951713 |
| Con-148 | 0 | 0.014915281 | 0.004688126 |
| Con-152 | 0 | 0.002630298 | 0.003828386 |
| Con-155 | 0 | 0.000566927 | 0.007671607 |
| Con-180 | 0 | 0.013068685 | 0.00275283 |

^{e}MLG: Metagenomic Linkage Group, defined as candidate species.
^{f}1 represents T2D group enrichment and bad marker; 0 represents control group enrichment and good marker.
g: The null hypothesis is that T2D groups don't differ from Control groups on the MLG, P value (P value < 0.05, considering as significant) means the probability of obtaining a test statistic at least as extreme as the one that was actually observed, assuming that the null hypothesis is true.

4.3 Prediction Analysis of Biomarkers

4.3.1 Prediction Analysis of Gene Makers

Using the gene relative abundances as the risk score, the inventors estimate the AUC (Michael J. Pencina, Ralph B. D'Agostino Sr, Ralph B. D'Agostino Jr, et al. Evaluating the added predictive ability of a new marker: From area under the ROC curve to reclassification and beyond. Statistics in medicine, 2008, 27(2): 157-172, incorporated herein by reference). The larger the AUC is, the more powerful the prediction ability on T2D disease is. For each gene, the inventors can estimate an AUC and its best cutoff where the sum of the prediction sensitivity and specificity reaches its maximum.

Detail of the cutoff: for a gene, the inventors first sort the samples' relative abundances. The inventors sequentially treat each relative abundance as the candidate cutoff and estimate its sensitivity and specificity. So the inventors can get the best cutoff on the maximal sum of the prediction sensitivity and specificity. For good gene, if the test sample's relative abundance is less than the best cutoff then the inventors predict the test sample is in disease condition. For bad gene, if the test sample's relative abundance is larger than the best cutoff then the inventors predict the test sample is in disease condition. See Table 4-1.

Sensitivity (also called recall rate in some fields) measures the proportion of actual positives which are correctly identified as such (e.g. the percentage of sick people who are correctly identified as having the condition). Specificity measures the proportion of negatives which are correctly identified (e.g. the percentage of healthy people who are correctly identified as not having the condition).

TABLE 4-1
AUC and CUTOFF of gene markers

| gene markers | Enrichment | | | | | sequence |
|---|---|---|---|---|---|---|
| ID | (direction) | cutoff | AUC | sensitivity | specificity | ID |
| 52049 | 0 | 6.44E−08 | 0.685 | 0.564706 | 0.752874 | 1 |
| 66281 | 0 | 3.63E−08 | 0.684 | 0.576471 | 0.741379 | 2 |
| 86279 | 0 | 3.06E−07 | 0.683 | 0.688235 | 0.626437 | 3 |
| 337304 | 1 | 1.18E−07 | 0.658 | 0.647059 | 0.632184 | 4 |
| 1224005 | 1 | 7.85E−08 | 0.666 | 0.611765 | 0.666667 | 5 |
| 1238449 | 0 | 1.18E−07 | 0.683 | 0.770588 | 0.568966 | 6 |
| 2005309 | 1 | 9.78E−08 | 0.657 | 0.635294 | 0.649425 | 7 |
| 2060779 | 0 | 4.15E−07 | 0.687 | 0.682353 | 0.666667 | 8 |
| 2370529 | 1 | 1.02E−07 | 0.66 | 0.641176 | 0.614943 | 9 |
| 2581190 | 1 | 3.10E−07 | 0.663 | 0.482353 | 0.804598 | 10 |
| 2746171 | 1 | 5.30E−08 | 0.659 | 0.705882 | 0.563218 | 11 |
| 3182475 | 1 | 1.25E−06 | 0.658 | 0.388235 | 0.873563 | 12 |
| 3247820 | 1 | 6.78E−08 | 0.662 | 0.605882 | 0.695402 | 13 |
| 3250057 | 1 | 3.09E−08 | 0.666 | 0.705882 | 0.568966 | 14 |
| 3253773 | 1 | 9.35E−08 | 0.657 | 0.682353 | 0.62069 | 15 |
| 3646621 | 0 | 4.60E−07 | 0.68 | 0.747059 | 0.563218 | 16 |
| 3793132 | 0 | 6.97E−07 | 0.681 | 0.723529 | 0.568966 | 17 |
| 3815768 | 0 | 8.22E−08 | 0.68 | 0.541176 | 0.775862 | 18 |
| 4097912 | 0 | 1.57E−07 | 0.689 | 0.652941 | 0.695402 | 19 |
| 4136092 | 0 | 1.20E−07 | 0.688 | 0.658824 | 0.683908 | 20 |

4.3.2 Prediction Analysis of Functions Makers

Using the functions maker relative abundances as the risk score, the inventors estimate the AUC (Michael J. Pencina, Ralph B. D'Agostino Sr, Ralph B. D'Agostino Jr, et al. Evaluating the added predictive ability of a new marker: From area under the ROC curve to reclassification and beyond. Statistics in medicine, 2008, 27(2): 157-172, incorporated herein by reference) The larger the AUC is, the more powerful the prediction ability on T2D disease is. For each functions maker, the inventors can estimate an AUC and its best cutoff where the sum of the prediction sensitivity and specificity reaches its maximum.

Detail of the cutoff: for a functions maker, the inventors first sort the samples' relative abundances. The inventors sequentially treat each relative abundance as the candidate cutoff and estimate its sensitivity and specificity. So the inventors can get the best cutoff on the maximal sum of the prediction sensitivity and specificity. For good functions maker, if the test sample's relative abundance is less than the best cutoff then the inventors predict the test sample is in disease condition. For bad functions maker, if the test sample's relative abundance is larger than the best cutoff then the inventors predict the test sample is in disease condition. See Table 4-2.

Sensitivity (also called recall rate in some fields) measures the proportion of actual positives which are correctly identified as such (e.g. the percentage of sick people who are correctly identified as having the condition). Specificity measures the proportion of negatives which are correctly identified (e.g. the percentage of healthy people who are correctly identified as not having the condition).

TABLE 4-2
AUC and CUTOFF of functions markers

| functions | Enrichment | | | | |
|---|---|---|---|---|---|
| markers | (direction) | cutoff | AUC | sensitivity | specificity |
| COG0229 | 1 | 5.03E−05 | 0.695 | 0.694118 | 0.643678 |
| K01251 | 1 | 5.84E−05 | 0.686 | 0.758824 | 0.517241 |
| K00162 | 1 | 1.01E−05 | 0.68 | 0.647059 | 0.655172 |
| K05396 | 1 | 2.33E−05 | 0.678 | 0.511765 | 0.798851 |
| K07315 | 1 | 5.39E−05 | 0.678 | 0.552941 | 0.747126 |
| COG0499 | 1 | 5.06E−05 | 0.674 | 0.782353 | 0.488506 |
| NOG134456 | 1 | 2.71E−06 | 0.674 | 0.441176 | 0.833333 |
| COG0659 | 0 | 0.000206 | 0.715 | 0.688235 | 0.678161 |
| K03321 | 0 | 0.000215 | 0.715 | 0.711765 | 0.66092 |
| K14652 | 0 | 0.000208 | 0.703 | 0.511765 | 0.827586 |
| NOG303876 | 0 | 2.37E−05 | 0.693 | 0.629412 | 0.706897 |
| K05339 | 0 | 0.000193 | 0.688 | 0.641176 | 0.649425 |
| COG1283 | 0 | 0.000369 | 0.679 | 0.670588 | 0.591954 |
| COG1266 | 0 | 0.000283 | 0.677 | 0.788235 | 0.494253 |
| K03324 | 0 | 0.000414 | 0.675 | 0.735294 | 0.528736 |

4.3.3 Prediction Analysis of Species Makers

Using the species maker relative abundances as the risk score, the inventors estimate the AUC (Michael J. Pencina, Ralph B. D'Agostino Sr, Ralph B. D'Agostino Jr, et al. Evaluating the added predictive ability of a new marker: From area under the ROC curve to reclassification and beyond. Statistics in medicine, 2008, 27(2): 157-172, incorporated herein by reference). The larger the AUC is, the more powerful the prediction ability on T2D disease is. For each species maker, the inventors can estimate an AUC and its best cutoff where the sum of the prediction sensitivity and specificity reaches its maximum.

Detail of the cutoff: for a species maker, the inventors first sort the samples' relative abundances. The inventors sequentially treat each relative abundance as the candidate cutoff and estimate its sensitivity and specificity. So the inventors can get the best cutoff on the maximal sum of the prediction sensitivity and specificity. For good species maker, if the test sample's relative abundance is less than the best cutoff then the inventors predict the test sample is in disease condition. For bad species maker, if the test sample's relative abundance is larger than the best cutoff then the inventors predict the test sample is in disease condition. See Table 5.

Sensitivity (also called recall rate in some fields) measures the proportion of actual positives which are correctly identified as such (e.g. the percentage of sick people who are correctly identified as having the condition). Specificity measures the proportion of negatives which are correctly identified (e.g. the percentage of healthy people who are correctly identified as not having the condition).

TABLE 4-1
AUC and CUTOFF of species markers

| | Enrichment | | | | |
|---|---|---|---|---|---|
| MLG ID | (direction) | cutoff | AUC | sensitivity | specificity |
| T2D-11 | 1 | 0.103658 | 0.618 | 0.541176 | 0.66092 |
| T2D-12 | 1 | 0.006279 | 0.654 | 0.564706 | 0.689655 |
| T2D-137 | 1 | 0.498151 | 0.585 | 0.423529 | 0.729885 |
| T2D-139 | 1 | 1.553228 | 0.617 | 0.5 | 0.701149 |
| T2D-140 | 1 | 0.49045 | 0.571 | 0.423529 | 0.735632 |
| T2D-14 | 1 | 0.010063 | 0.652 | 0.764706 | 0.505747 |
| T2D-154 | 1 | 8.95E−05 | 0.604 | 0.411765 | 0.798851 |
| T2D-15 | 1 | 0.00508 | 0.589 | 0.670588 | 0.494253 |
| T2D-165 | 1 | 0.032528 | 0.6 | 0.488235 | 0.701149 |
| T2D-16 | 1 | 0.003242 | 0.634 | 0.6 | 0.626437 |
| T2D-170 | 1 | 0.032845 | 0.616 | 0.417647 | 0.804598 |
| T2D-1 | 1 | 0.098314 | 0.526 | 0.076471 | 0.977011 |
| T2D-2 | 1 | 0.0072 | 0.586 | 0.388235 | 0.816092 |
| T2D-30 | 1 | 0.001567 | 0.54 | 0.147059 | 0.936782 |
| T2D-37 | 1 | 0.099862 | 0.591 | 0.411765 | 0.770115 |
| T2D-57 | 1 | 0.015788 | 0.647 | 0.523529 | 0.701149 |
| T2D-5 | 1 | 0.000673 | 0.651 | 0.688235 | 0.563218 |
| T2D-62 | 1 | 0.274395 | 0.624 | 0.417647 | 0.793103 |
| T2D-6 | 1 | 0.089696 | 0.526 | 0.094118 | 0.982759 |
| T2D-73 | 1 | 0.107684 | 0.6 | 0.311765 | 0.885057 |
| T2D-79 | 1 | 0.150142 | 0.572 | 0.594118 | 0.563218 |
| T2D-7 | 1 | 0.046154 | 0.604 | 0.523529 | 0.655172 |
| T2D-80 | 1 | 0.003178 | 0.655 | 0.682353 | 0.586207 |
| T2D-8 | 1 | 0.007389 | 0.622 | 0.641176 | 0.58046 |
| T2D-90 | 1 | 0.009561 | 0.62 | 0.447059 | 0.758621 |
| T2D-93 | 1 | 0.034981 | 0.563 | 0.417647 | 0.718391 |
| T2D-9 | 1 | 0.008346 | 0.62 | 0.570588 | 0.637931 |
| Con-101 | 0 | 0.011503 | 0.672 | 0.717647 | 0.58046 |
| Con-104 | 0 | 0.156174 | 0.668 | 0.658824 | 0.632184 |
| Con-107 | 0 | 0.34953 | 0.656 | 0.652941 | 0.637931 |
| Con-109 | 0 | 0.001797 | 0.641 | 0.423529 | 0.816092 |
| Con-112 | 0 | 0.059392 | 0.606 | 0.529412 | 0.632184 |
| Con-113 | 0 | 0.36604 | 0.646 | 0.641176 | 0.614943 |
| Con-120 | 0 | 1.686662 | 0.62 | 0.705882 | 0.5 |
| Con-121 | 0 | 0.06585 | 0.67 | 0.688235 | 0.568966 |
| Con-122 | 0 | 0.003649 | 0.602 | 0.723529 | 0.448276 |
| Con-129 | 0 | 0.663083 | 0.618 | 0.658824 | 0.557471 |
| Con-130 | 0 | 0.403354 | 0.604 | 0.664706 | 0.54023 |
| Con-131 | 0 | 0.639878 | 0.643 | 0.6 | 0.62069 |
| Con-133 | 0 | 0.419924 | 0.627 | 0.717647 | 0.505747 |
| Con-142 | 0 | 0.180048 | 0.625 | 0.529412 | 0.655172 |
| Con-144 | 0 | 0.082044 | 0.613 | 0.564706 | 0.649425 |
| Con-148 | 0 | 0.689789 | 0.605 | 0.758824 | 0.408046 |
| Con-152 | 0 | 0.222946 | 0.598 | 0.705882 | 0.494253 |
| Con-155 | 0 | 0.001098 | 0.575 | 0.811765 | 0.321839 |
| Con-166 | 0 | 0.001912 | 0.67 | 0.5 | 0.781609 |
| Con-180 | 0 | 7.74E−05 | 0.599 | 0.694118 | 0.494253 |

### Example 5

### Rebuilt Microbial Genomes Associated with Diseases

5.1 Advanced-Assembly

Use the method in Example 3 to conduct MLG advanced-assembly rebuilt microbial genomes associated with diseases (results shown in Table 6)

TABLE 6
MLG advanced-assembly

| MLG ID | Assembled size (bp) |
|---|---|
| T2D-154 | 1,459,858 |
| T2D-140 | 306,933 |
| T2D-139 | 4,076,917 |
| T2D-11 | 5,461,429 |
| T2D-5 | 5,685,283 |
| T2D-80 | 3,343,701 |
| T2D-57 | 2,235,135 |
| T2D-15 | 4,343,101 |
| T2D-1 | 1,147,560 |
| T2D-7 | 1,475,127 |
| T2D-137 | 360,515 |
| T2D-165 | 382,494 |
| T2D-12 | 1,279,239 |
| T2D-8 | 6,360,192 |
| T2D-93 | 4,013,431 |
| T2D-62 | 3,110,163 |
| T2D-2 | 5,468,995 |
| T2D-6 | 2,046,892 |
| T2D-9 | 177,039 |
| T2D-14 | 336,706 |
| T2D-16 | 514,526 |
| T2D-30 | 1,160,723 |
| T2D-37 | 425,200 |
| T2D-73 | 1,058,177 |
| T2D-79 | 2,324,477 |
| T2D-90 | 202,401 |
| T2D-170 | 349,106 |
| Con-107 | 2,425,544 |
| Con-112 | 625,210 |
| Con-129 | 2,763,410 |
| Con-166 | 300,056 |
| Con-121 | 3,263,915 |
| Con-113 | 912,962 |
| Con-120 | 329,961 |
| Con-130 | 1,777,506 |
| Con-131 | 712,548 |
| Con-133 | 2,336,766 |
| Con-109 | 864,710 |
| Con-101 | 2,209,191 |
| Con-104 | 1,430,920 |
| Con-122 | 2,764,146 |
| Con-142 | 1,366,065 |
| Con-144 | 317,022 |
| Con-148 | 1,910,062 |
| Con-152 | 1,794,972 |
| Con-155 | 514,244 |
| Con-180 | 2,060,466 |

5.2 Identification of Microbial Genomes

Use the method in Example 3 to conduct MLG taxonomic assignment based on the obtained microbial genomes (results shown in Table 7)

TABLE 7
MLG taxonomic assignment

| | | Number | | | |
|---|---|---|---|---|---|
| Enrichment | MLG ID | of genes | Taxonomy assignment (level) | % genes^{h} | similarity ⁱ |
| T2D group | T2D-154 | 337 | Akkermansia muciniphila | 97.92 | 98.17 ± 0.09 |
| enrichment | T2D-140 | 148 | Bacteroides intestinalis | 89.19 | 98.20 ± 0.15 |
| | T2D-139 | 3,386 | Bacteroides sp. 20_3 | 94.60 | 99.29 ± 0.01 |
| | T2D-11 | 5,113 | Clostridium bolteae | 96.87 | 99.39 ± 0.02 |
| | T2D-5 | 2,378 | Clostridium hathewayi | 96.93 | 99.31 ± 0.03 |
| | T2D-80 | 2,381 | Clostridium ramosum | 95.38 | 99.81 ± 0.01 |
| | T2D-57 | 821 | Clostridium sp. HGF2 | 97.69 | 99.59 ± 0.03 |
| | T2D-15 | 2,492 | Clostridium symbiosum | 95.63 | 99.58 ± 0.01 |
| | T2D-1 | 949 | Desulfovibrio sp. 3_1_syn3 | 93.78 | 98.04 ± 0.08 |
| | T2D-7 | 1,056 | Eggerthella lenta | 94.22 | 99.63 ± 0.03 |
| | T2D-137 | 425 | Escherichia coli | 70.35 | 99.01 ± 0.08 |
| | T2D-165 | 131 | Alistipes (genus) | 89.31 | |
| | T2D-12 | 364 | Clostridium (genus) | 79.40 | |
| | T2D-8 | 5,272 | Clostridium (genus) | 65.35 | |
| | T2D-93 | 1,590 | Parabacteroides (genus) | 60.69 | |
| | T2D-62 | 2,584 | Subdoligranulum (genus) | 93.81 | |
| | T2D-2 | 2,430 | Lachnospiraceae (family) | 95.43 | |
| | T2D-6 | 1,305 | Unclassified | 96.55 | |
| | T2D-9 | 105 | Unclassified | 67.62 | |
| | T2D-14 | 392 | Unclassified | 74.74 | |
| | T2D-16 | 222 | Unclassified | 72.07 | |
| | T2D-30 | 430 | Unclassified | 98.84 | |
| | T2D-37 | 251 | Unclassified | 92.03 | |
| | T2D-73 | 565 | Unclassified | 96.81 | |
| | T2D-79 | 1,632 | Unclassified | 86.89 | |
| | T2D-90 | 130 | Unclassified | 99.23 | |
| | T2D-170 | 114 | Unclassified | 95.61 | |
| control | Con-107 | 1,677 | Clostridiales sp. SS3/4 | 97.02 | 97.95 ± 0.06 |
| group | Con-112 | 232 | Eubacterium rectale | 90.52 | 97.56 ± 0.12 |
| enrichment | Con-129 | 1,440 | Faecalibacterium prausnitzii | 96.74 | 98.18 ± 0.04 |
| | Con-166 | 273 | Haemophilus parainfluenzae | 95.24 | 94.81 ± 0.17 |
| | Con-121 | 3,507 | Roseburia intestinalis | 92.19 | 98.90 ± 0.03 |
| | Con-113 | 345 | Roseburia inulinivorans | 94.20 | 98.21 ± 0.11 |
| | Con-120 | 116 | Eubacterium (genus) | 55.17 | |
| | Con-130 | 670 | Faecalibacterium (genus) | 51.94 | |
| | Con-131 | 202 | Faecalibacterium (genus) | 77.23 | |
| | Con-133 | 1,555 | Erysipelotrichaceae (family) | 77.88 | |
| | Con-109 | 378 | Clostridiales (order) | 74.87 | |
| | Con-101 | 1,762 | Unclassified | 85.70 | |
| | Con-104 | 916 | Unclassified | 67.58 | |
| | Con-122 | 1,999 | Unclassified | 80.24 | |
| | Con-142 | 673 | Unclassified | 95.39 | |
| | Con-144 | 162 | Unclassified | 96.91 | |
| | Con-148 | 481 | Unclassified | 82.95 | |
| | Con-152 | 945 | Unclassified | 81.16 | |
| | Con-155 | 228 | Unclassified | 89.47 | |
| | Con-180 | 528 | Unclassified | 86.55 | |

^{h}percentage of MLG genes in the closest species
ⁱ average similarity of the closest species

### Example 6

### Odds Ratios of Species Markers

In order to further verify the found species markers, the odds ratio of each species marker was calculated in the 344 samples above (shown in Table 8). The results showed that the species have high strength association (Odds ratio is greater than 1. Greater odds ratio is, more obviously enriched in the corresponding group of samples the species marker is).

TABLE 8
odds ratios of species markers

| | | Taxonomy assignment | Odds ratios | |
|---|---|---|---|---|
| Enrichment | MLG ID | (level) | (95% CI) | |
| T2D group | T2D-154 | Akkermansia muciniphila | 1.52 | (1.05, 2.19) |
| enrichment | T2D-140 | Bacteroides intestinalis | 1.50 | (1.15, 1.97) |
| | T2D-139 | Bacteroides sp. 20_3 | 1.66 | (1.26, 2.20) |
| | T2D-11 | Clostridium bolteae | 5.89 | (1.39, 25.0) |
| | T2D-5 | Clostridium hathewayi | 23.1 | (2.08, 256.6) |
| | T2D-80 | Clostridium ramosum | 1.68 | (0.97, 2.89) |
| | T2D-57 | Clostridium sp. HGF2 | 2.62 | (1.14, 6.03) |
| | T2D-15 | Clostridium symbiosum | 1.13 | (0.88, 1.44) |
| | T2D-1 | Desulfovibrio | 1.41 | (0.93, 2.13) |
| | | sp. 3_1_syn3 | | |
| | T2D-7 | Eggerthella lenta | 1.57 | (0.95, 2.58) |
| | T2D-137 | Escherichia coli | 1.72 | (1.16, 2.57) |
| | T2D-165 | Alistipes (genus) | 1.46 | (1.07, 1.99) |
| | T2D-12 | Clostridium (genus) | 2.22 | (1.12, 4.40) |
| | T2D-8 | Clostridium (genus) | 1.12 | (0.86, 1.45) |
| | T2D-93 | Parabacteroides (genus) | 1.84 | (1.03, 3.29) |
| | T2D-62 | Subdoligmnulum (genus) | 2.41 | (1.43, 4.08) |
| | T2D-2 | Lachnospiraceae | 4.06 | (1.28, 12.9) |
| | | (family) | | |
| | T2D-6 | Unclassified | 3.70 | (1.18, 11.7) |
| | T2D-9 | Unclassified | 1.02 | (0.83, 1.27) |
| | T2D-14 | Unclassified | 9.61 | (1.93, 47.8) |
| | T2D-16 | Unclassified | 1.17 | (0.87, 1.56) |
| | T2D-30 | Unclassified | 1.27 | (0.94, 1.73) |
| | T2D-37 | Unclassified | 1.68 | (1.27, 2.22) |
| | T2D-73 | Unclassified | 1.89 | (1.26, 2.83) |
| | T2D-79 | Unclassified | 1.28 | (0.97, 1.68) |
| | T2D-90 | Unclassified | 2.01 | (1.29, 3.13) |
| | T2D-170 | Unclassified | 1.85 | (0.96, 3.57) |
| control | Con-107 | Clostridiales sp. SS3/4 | 1.44 | (1.13, 1.84) |
| group | Con-112 | Eubacterium rectale | 1.51 | (1.13, 2.03) |
| enrichment | Con-129 | Faecalibacterium | 1.55 | (1.19, 2.00) |
| | | prausnitzii | | |
| | Con-166 | Haemophilus | 1.25 | (0.93, 1.69) |
| | | parainfluenzae | | |
| | Con-121 | Roseburia intestinalis | 3.10 | (1.92, 5.03) |
| | Con-113 | Roseburia inulinivorans | 1.45 | (1.11, 1.89) |
| | Con-120 | Eubacterium (genus) | 1.55 | (1.17, 2.06) |
| | Con-130 | Faecalibacterium (genus) | 1.59 | (1.21, 2.08) |
| | Con-131 | Faecalibacterium (genus) | 1.58 | (1.16, 2.15) |
| | Con-133 | Erysipelotrichaceae | 1.52 | (1.15, 2.01) |
| | | (family) | | |
| | Con-109 | Clostridiales (order) | 1.41 | (1.09, 1.83) |
| | Con-101 | Unclassified | 1.56 | (1.00, 2.43) |
| | Con-104 | Unclassified | 1.96 | (1.33, 2.89) |
| | Con-122 | Unclassified | 1.97 | (1.16, 3.34) |
| | Con-142 | Unclassified | 1.38 | (1.03, 1.83) |
| | Con-144 | Unclassified | 1.38 | (1.09, 1.74) |
| | Con-148 | Unclassified | 2.10 | (1.31, 3.36) |
| | Con-152 | Unclassified | 1.53 | (1.17, 2.00) |
| | Con-155 | Unclassified | 1.72 | (1.18, 2.50) |
| | Con-180 | Unclassified | 1.64 | (1.15, 2.32) |

TABLE 9
Sequences of gene markers

>52049
ATGTACATTGCAGATGGAAAAACAAACGGACCAGCGTTTTCCTGGCCAGACGGCAAACGCATTGCCGTGATGGTT
ACATTTGATTATGACGCTGAATTTTTACGGATATCCCGCGCCAAAAGCAAGGGAACGAGCATTGGCTTTACCGAT
TTTTCACGAGGCCAGTACGGCCCCCATGAGGGACTGGCCAGATGCCTTGATATGTTAGATACCATGAACATCAAA
TCTACCTTTTTTGTGCCGGGCGCTGTGATCGAGACCTACCGGGATACCGTAGAGGAAATCCACCGGCGCGGCCAT
GAGCTGGCCTGCCACGGCTACCGGCATGAATCCGATCCGGAGCTTTCCCGGGACGAAATGATAAAAATCCTGGAT
AAAAGTGAAGCGCTGCTCGCAGAGATCACCGGAAAAAAGCCAGTAGGACACCGGGCACCGGAAAGCGTGCTCCAG
GATTTTATGCCGGAGCTTCTGGCTGAGCGTGGCTATCTGTACAGTTCTTCCATGAAGGACTGTGACTGGGCTTAT
CTCTGGGAAAAGGATCAAAAGGAGCTGCCCCTGGTAGAGCTCCCAAACGATATCACCATGGATGACTTTACCTAT
TACTATTTCACCTTCAGTGATCCTGCAGTCCGCTGTATGTACCCGAACCGTGAGGTCTTCGGCAACTGGAAGCAG
GAATTTGACGGTCTGGCCCTGGAGGGCAACAAGATCTTCATCTTAAAGCTGCATCCGCAGATGATCGGCCGCGCG
AGCCGCATCGGCATGGTAGGCGAATTCATTGCCTACATGCAGAATCACGGCGCATGGATCACCACCTGCGAGGAT
GTAGCACGTTATGTACAGAAGCAGAACGGAGGAAACAGAGCATGA
>66281
ATGATCCGGAAACGTGCAAAACGGCTGGCAGGCGCAGTGCTTGCCGCCGGCGCCATTACGGCAATGCCATTTCAG
GCATTTGCCCAGAGAAGCCCGGAATTTGCTTATTCAGCAGAAAAATGGGCGACACTCCGCGACAATAAGCTGGAG
TTTGATGAGATTTCAGATCTGGTTCATGAGTACAATCCGACCGTGGTCCAGAACGAGATCAGCTACAAGGATTAT
CTGACCAAGAACCGGGATGATGTGGCCCAGGATTATTACGACAAGGCCAATGAGATTTATTCCAATATCAGCTAC
CCGGACTCAGATGATGCCAACTACGGAAGTGGTGTGGCAGCGGCACTGCGCAATGAACAGCAGGCCAAAAGCCTG
ATGGAGCAGGGTGATGAAAACACCGATGACCAGGCCACCATGCGGATCCAGTACGATCAGGCCGAGGCGAAGCTG
GCCAAGCAGGCACAGGGGCTTATGATCACCTACTGGACCCAGTACTATAACCTGGATGGCCAGAAGGCCCGCGTT
GAGCAGGCGAAGCTTTCGTACCAGTCTGAGCAGAACCGCCTGGCAGCAGGGATGTCTACCCAGTCCAAGGTTTTA
AGCGCAAAGGAGTCCGTCTCCAATGCGGAGGCAGCGCTGGTGACTGCCGAGAGCAATCTGGCCTCCACAAAGGAG
AGTCTGTGCCTGATGTTAGGCTGGGGCTACGGCGCGGATGTGGAGATCGCGGAGCTTGCAGAACCGGACCAGAGT
AAGATCGCGGCCATTGATGTGAATGCGGATATCCAGGCAGCTCTGGAGAACAGCTACGCCTACCGCTTGACGAAA
AAGCAACTCACCAACGCCAGAACAGACAGCGTGAAGGATAAGCTGAGCGAGACGGAAAAGAATCAGAGGGAGACC
ATCTCCAACAGTGTGAAATCTGCTTATGATTCCCTGCTTCTGGCTCAGTCCGGTTACGAGCAGGCTCAGTCCGCG
CTGGCGCTGCAGGAGGTTTCCATGAAGTCTGTCGACGCGAAGCTGGCAGCGGGAACCATTACAAAAAATACCTAT
GAGAGCCAGAAGGCATCCTACACCACCGCCCAGGTGACTGCCCAGACCCAGAAGCTGTCCCTGTTACAGGCCATG
AATGATTATGACTGGGCCGTGAACGGACTGGCATCTGCAGAGTAA
>86279
ATGGCAGAGAATATTTTACAGGTAAAAAATTTAAAAACCTACTTTCATACTGAGGCCGGACTTGTGAAAGCGGTT
AACGATGTTTCCTTCAATGTGGAAAAGGGTAAGACCCTCGGCATTGTAGGTGAGTCCGGCTGCGGAAAAAGTATC
ACTTCCTTATCGATCATGGGTCTGGTAGAGCGTCCCGGTAAGATCGAGGGCGGTGAGATCCTGTTTGAGGGGGAA
GACCTCTTAAAGATGACGGAGGCTCAGATGCGCAACATCCGCGGCAAGAAGATTGCCATGATCTTCCAGGAGCCG
ATGACATCCTTGAATCCGGTTTATACCATCGGACAGCAGCTGATCGAGGCCCTGCTCCTTCATGAGAAAATGACC
AAGCAGGAAGCAAAAGCCCGCGCCATCGAGATTTTAAAGCTGGTTAAGATCCCGCTTGCGGAGCGCCGCTTCGAC
GAGTACCCGCATCAGCTTTCCGGCGGTATGCGTCAGCGTGTCATGATTGCCATGGCACTCTGCTGTAATCCGGCT
ATGCTGATCTGTGATGAGCCGACTACTGCGCTGGACGTAACCATTCAGGCGCAGATTCTGGACCTCATCAATGAG
TTAAAAGAAAAGACCGGAACCTCCGTTATGATGATTACCCACGACCTGGGTGTCATCGCAGAGGTTGCGGATGAT
GTTATGGTTATGTACGCAGGCAAGGTCGTTGAGCACGCAAGCTGCGACCAGATCTTTGACAAGCCGATGCATCCG
TATACGGACGGACTGATGAAGTGCATTCCGAAGCTGGATGATGACGACACCAAAGAGCTGAGCGTTATCAAGGGC
ATGGTGCCAAGCTTTGATGATATGCCGGCAGGCTGCGCGTTCTGCCCGAGATGTCCGCAGGCCCGCGAGATCTGC
CGTCAGAAGATGCCGGAGTTAGTCGAAGCCGAGGGCCGCAAAGTGCGCTGCTTTAAATATACGAAGGAGTGGGAG
GAGAACATCTGA
>337304
ATGGAGCTTTTAAAACAGCGGATCCTGCGGGACGGGCAGGTGAAAGAAGGCGGCGTACTCAAGGTGGACAGCTTT
CTCAACCACCAAATGGATGTGACGCTGCTGAATGAGATCGGGCGTGAATTCCGCCGCCGTTTCGACGGAGCGGCC
ATCACCAAAATCGTTACCATCGAGGCGTCCGGCATCGGTATTGCCGCCATTGCCGCGCAGCATTTCGGCAGCGTT
CCCGTCATCTTCGCCAAAAAGACGCGTTCGCGCAATCTGGACGGTGCGCTGTATACCGCCAAGGTCCATTCTTTC
ACCAAGGACATTACCTACGATGTGCAGCTATCCAAAAAATTTCTCGGCCCGCAGGACACTGTGCTCATCCTGGAC
GATTTCCTCGCCCGGGGACAGGCGCTGCTGGGCCTCATCGACATCGTGCGGCAGGCGGGCGCGCAGTGCGCTGGC
TGCGGCATCGTCATTGAAAAGCAGCAGCAGGGCGGCGGCGCGCTGGTGCGGGCAGCGGGAGTGCGCCTGGAATCG
CTGGCCGTAATCGCCTCGCTGGAAAACGGCACGGTCACCTTTGCGCAGTGA
>1224005
ATGGCGGACTGCCCTATTAACTCAGCCTCAGAGACTATTGCTGACTTTATCTACCGTAATGGAAGCCAGCAGCAA
TTTCCTGGTAATTCGGAAGATATTCCCTGTATGGACTTTGTAAGCACTGATTTCAGCATTATATATACCCCGCTA
GATACCGTAGAACCCATATCACTAAGTAAGTTTACCTATTATAGTATTCCTGGACTGTACACACTGTTAGATAGT
TCCAGCATGGACGCTTCCGGCATTTTGGCAACCCATGCATCACCTGCCCTTAGCAATCAAGGGAGAGGTGTTATA
ATCGGCATCATAGACACGGGAATTGACTATACCAATCCTTTATTTCGTAATCAGGATGGAACTACCAGAATCTTG
AGTATTTGGGATCAGAGCCTTCCAGAAGATAAAAGCCTCCTTCCTGCCGGTGTACCCAACCGCTATAATGCCAGC
GGGGCAAGCTATGGCACAGAGTACACGCAGGAGCAGATTAACGAGGCACTGGAATCTGATAATCCGTTTGCCGTG
GTGCCTTCTACCGATACCAACGGACATGGAACCTTTTTGGCGGGTATCGCTGCTGGAGGAATCCTGCCTAATCAG
GATTTTACAGGAGCAGCACCGGAATGTGAACTGGTAATCGTCAAATTAAAACCAGCCAAACAATATCTGCGCGAT
TTCTATCTTATCAGTAATGATGCGGATGCTTATCAGGAGAATGATATAATGATGGGCATTAAGTACCTGCGTGTG
GAGGCATACAGCCAGAGAAAACCTCTGGTTATTTTACTGGGAATCGGCTCCAATCTGGGCAGCCATGAAGGGACG
TCTCCATTGAATGTAATGATTCAAGATATCAGCCGATATTTAGGTATGGCTACGGTAATTGCTGCAGGCAATGAA
ACTGGCCGGGGGCATCATTACATGGGGTCTGTACCGATAGGGGAAGAGTCCGTTGAGGTGGAAATCCGAGTAGGG
AATGCGGAATCCCAACGTGGATTTGTAGTAGAACTCTGGGCCGACACCGCTGATACATATTCGGTTGGATTTGTC
TCACCCAGTGGTGAATCCATAGGCCGCATTCCTATCATAGGCAGAAACGAAACCTCTATCCCGTTCCTTTTGGAA
CCCACGGTCATTACAGTTAATTACCAGCTGATTGAGACAGGCGCAGGCAAACAGCTTGTTTTTATCCGTTTTGCA
GCCCCAACTAATGGAATTTGGAGAATACGCGTGTATAATACCCAGTATCTTACAGGGCAATTTAATATGTGGCTT
CCGGCCCATACCTTGATTTCTGATGAAACCGTGTTTCTGACTCCCAGCCCATACACGACCATAACCCTCCCGGGA
GATTCTCCCTCTCCAATCACAGTAGGAGCCTATAATCATTTAAATAACAGCATCTACATTCATTCCGGCCGTGGA
TATACAATAGGCGGATTGATAAAGCCGGATTTGGCAGCTCCAGGGGTTAATGTAAGCGGGCCATCGATAGGGCAA
AGGAACCAAGACTCCATTCCCATGACTACCCGAACCGGAACCTCGGTTGCTGCTGCCCATGTTGCTGGTGCAGTC
GCTAATCTTTTAAGCTGGGGCATAATAGAAGGACATAACATAGCAATGAGCGAAGCCACTGTCAAAGCATTTTTA
ATAAGAGGAGCAAAACGGAATCCAGCACTATCATATCCCAATAGGGAATGGGGATATGGGGCTCTGAACTTATAT
GAAACTTTTTTACGGTTAAGAGAAATAAGATGA
>1238449
TTGGATTACTTAGTATTAACGAAGGTTCATGGGGCGATTCTTGGTCCGATCTCCCAGATTCTGGGATGGATCCTG
AATGTACTCGTTACGTTCACCAATTCCTTTGGTGTACTTAACATCGGTTTATCCATTATTTTATTTACCCTGGTT
GTGAAATTACTGATGTTCCCTATGACGATCAAGCAGCAGAAGGCATCCAAGCTGATGGCTGTCATGCAGCCTGAG
CTTCAGGCTATTCAGGCAAAGTATAAGGGCAAAACAGACAATGACTCCATGATGAAGATGAACGTGGAGACAAAA
GCTGTCTATGAGAAATACGGCACATCCATGACTGGAGGATGTCTCCAGCTTGTGATCCAGATGCCGATCCTGTTT
GCGCTGTACCGCGTGATCTATTGCATTCCGGCTTACGTGATGCCGGTAAAGGAACATTTTCTGAATGTGGTCTAT
GCACTGACCGGAACTTCTTCCGCGTCTGCCCTGAGCGAGGGGGCTGCGGCAAATCTGCTCCAGTTTGCGACCGAC
CACAATATTGCGTTAACGGGCATTAACCCGATCGGAGACTTAACCGGTGTGAACGGTGAGGCTCTGGCCAATAAG
ATGGTTGATATTCTCTACAAGCTGAATCCGGCTCAGTGGAATGATCTGGCGCAGGCATTCCCGAATGCGGCAGAT
GTCATTTCCACAAATGCGTCTACCATTGAGAAGATGAATACCTTCCTTGGCATTAATCTGGCATCCAACCCGTTT
AACGGAAGCTTTGTACCGAGTCTGGCATGGCTGATTCCGATTCTGGCAGGTCTGACCCAGTATGCGAGCACAAAG
TTAATGATGGCAACCCAGCCGAAGAAGAACAATGAGGAAGATATGAGCTCCCAGATGATGCAGAGCATGAACGTT
ACCATGCCGCTGATGTCTGTATTTTTCTGCTTTACCTTCCCGGCAGCAATCGGTATCTACTGGGTAGCCAGCAGT
ACCTTCCAGTTACTGCAGCAGCTTGTTGTAAATTCTTACCTGGATAAGGTAGATATGGATGAGCTGATCCGTAAG
AACGTTGAGAAGGCTAACAAGAAACGCGCAAAGAAAGGCCTTCCGCCGCAGAAAGTAAATCAGAACGCGACCGCA
AGCTTAAAGCATATGCAGGCAGTTCAGGAGAAGGAAGAAGCGGAACGCGCTGAGAAGCTTGAGAAGAGCAAAAAA
CAGGTAGAAGCATCCAACAATTATTATAATAAAGATGCAAAACCGGGCAGTCTGGCCTCCAAGGCAAATATGGTA
GCCAGATATAATGAAAAGCACAATAATAAATAA
>2005309
ATGGAAAAAATTCACACGGAAAAGGCCCCGGCCGCAATCGGCCCTTATTCGCAGGCGATGAAGCAGGGGGGGCTG
GTGTTCACCTCCGGCCAGATCCCGCTGGACCCGGCGACGGGCGCCGTCGTGGGGGAAGATATCACAGCGCAGGCA
AAGCAGGTGCTTGAAAACCTCAAGGCCGTGCTGGAAGCATCCGGTGCGGCGCTGGATACAGTGCTCAAGACGACA
TGCTTTTTGGCGGATATGGCGGACTTCGCGCCCTTCAACGAGCTGTATGCGGCCTATTTCACAGGCTGCCCGGCA
CGCTCCTGCTTGGCGGTGAAACAGCTGCCCAAAGGTGTGCTCGTAGAAGTGGAGGCCATTGCGGCGGTGCGCTGA
>2060779
ATGAAAAATTATACAATCACAGTAAACGGTAATGTATATGAAGTAACAGTAGAAGAGGGCTTCACAGGCGCAGCT
TCCGCACCGAAGGCAGCAGCACCGGCACCGAAGGCAGCACCGGCAACAGCACCGAAGGCAGCTCCGGCACCGGCA
GCAGCTCCGGCAGCTCCGGCTGGCGCAGCAGGCGCAGTAGCAGTTACAGCTCCGATGCCGGGTAAGATCCTTGGC
GTTAAGGCATCTGCAGGTCAGGCTGTTAAGAGAGGTCAGGTTCTCCTGATCCTTGAAGCTATGAAGATGGAGAAC
GAGATCGTTGCTCCGCAGGACGGCACTGTTGCAACAATCAACGTTGCTGTTGGTGATTCCGTAGAGCCGGGTGCT
ACACTCGCTACATTAAACTAA
>2370529
TCTGTATTTCAAGTTGCTTTCCACAGCTTTCGCTGTTACAATAAGCCTATCGAAGAAACGGAGGTCCTCATGAAG
ATCATTTTCGTCTGGCTCCTTGCCATTGTTTTCCTCATCGACAGCGTCCTGCGCGCCATCCGCTCCAGCTTCAAC
CTTGGGGTGCTGATGATGTATCTCATCACTGCCGCACTGTGGATATACGCTCTTTTCCATACCAAAATCGACGCT
TTTTGCGCTGCCGGTGCAGGCCGCGTTCTAAAAATCATCTTTTTCTGCTGCTGCGCCGTTTTTGCGCTGCTGCTT
ATATTCGTCGCGGTGAGCGGCTACTCCGACACCGCGACCAAGCAGGAAAAAGCGGTGATCGTACTGGGCGCCGGC
CTGCGAGGCGAACGCGTGACCGACCTTTTGGCACGCCGTCTGGATGCCGCATATGATTATCATCTGGAAAACCCG
AATGCCGTTATTGTTGTGACTGGCGGACAGGGTCCCGGCGAGGACATTCCCGAAGCCCGGGCCATGAAAGCCTAT
CTTGTGGAGAAAGGCGTGCCGGAGAAGCAAATTCTGGAAGAAGCGTCCAGTACCTCCACCGAGGAAAATTTTTGC
TTTGCGCGCGAAATTTTGGAACAGCACGGTCTTTCGCAGGACGAACCCGTCGCGTATGTCACCAATGCGTTCCAT
TGTTACCGCGCAGCAAAATACGCTGCCGCCGCAGGCTTCACAAATGTGAACGCCACCCCCGCCTCTATCGGCTTT
TCTTCCGTACTCCCCTGTTATATGCGTGAGGTAATGGCGGTGCTGTTACTACTGGATGTTTCGCACCTGA
>2581190
TCCAGCGAACGGAAGGATTTCATCATTGTTTCCGAGCGCAGACCATATCCGATGAACCCGATGCGTTTCATAATT
CCATTCCTCCATTTTTTGTGCGGGGCCGCTTTTCTTTATATTACATATAAAAGGCGGGCAAATGGCTTTGCAGTG
CGCCTGCGCCGCTTTTTATACGCTCGCTGCGCTAATAATTATTTTGTTATTAGCAAATTTTAA
>2746171
ATGATAATTGATTTGAGCCTTCAGCAGTATGTGGATTGGGAAGTAAAGCCAGTGATTCCTATAAAGGGAAAATTC
GGATACCGTGTTGTGTTAAAATACATAGATGGTACTGAAAGAACTCAGCAAAAGTCGGGGTTTAGGACAGAAAAA
GAGTCTAATGCTGCCAGGGATAACACAATAGGCAAATTACATGCTGGAACTTATATTGTATATGAGAATGTTAAA
GTAAGTGACTTTTTAGAGTTTTGGCTAAAGGAGGATATATGCAAGAGGGTAAGAAGCGAAGAAACTTATGCTGCT
TATTCCAATATTGTGTATAACCACATTATTCCAATCCTTGGAAAGAAAAAGATGTCGGCCGTTAACCGTGGAGAT
GTTCAAAAGCTGTATAACGACCGGGCAGAATATTCTGTATCGGTCTCAAGGCTGGTTAAAACGGTGATGAATGTC
TCTATGAACTATGCTGTAGCGAAAAAAATCATTGCGGAGAATCCGGCAGTTGGTATCAATCTTCCTAAAACGGTA
AAGAAGAAGGAATACCATGCCCGCAGTATTGACACACAGAAAACCCTGACAATGGATCAGATTCTGATTCTATTG
GAGGCCAGCAGGGACACGCCGATACATATGCAAATCCTGCTTATGTATTAATGGGGACTTCGCAGAAGGGAAATC
AACGGTGTGAAATATTCGGATATTGACTATATTAACCGAACACTAAAGCTTCGCCGGCAGTTGGGGAAGAAAATC
AACACAAAAAAAGAAGACTTTCCACCTAAAACCTTTACGAAACAGGAACTTGGATTGAAGACCCCATCGAGCTAT
CGTGATATTCCGATACCAGATTATGTGTTTGAGGCGATTCTGCAACAGAGGGAGGTGTATGAGAGAAATAAGAGT
CGCAGGAGAAGCCAGTTTCAGGATTCAGGATATGTTTGCTGCTCTAACTACGGGAAACCGCGAAGCAAGGATTTC
CATTGGAAATATTATAAAAAACTGTTAGCGGATAACAACCTGCCGGATATAAAGTGGCATCATTTACGCAGTACC
TTCTGTACACTGCTTTTGAAAAATAATTTTAATCCCAAAGCGGTATCCAAATTAATGGGACATGCAACGGAGCTA
ATCACCTTAGATGTATATGGTGATAACCGGGAAATTATTGCTGACTGTGTAGATGAAATTCAGCCGTTCATAGAT
GAGGTGCTTCCGATAAAGGAGGTAGACAAGCAGCTTGAGGAAGAACTGTTGGAAATCGAAGTTCCGGCAGAAGAT
TATGTTTAA
>3182475
TTTCCGAAAGGGAAGGACTCTCCAAAGCGTCGTACCTTGAAAACTGAACAAAGACTGCGTAAAGACAAAAGGATG
GATGTTCCACCAGGAAGCATTCATCATTTGAAAGAGATTTTAAAAGAAATCTACAATTTCAAAGTTCTTTTGTTG
GCAAATGGAAGAATCGAGCAAGAAAGATAA
>3247820
TTGCTAAAAAAAGAAAAGGAGAAAGAAAAAATGAACATGGAAACGTGTAAGACAAGTTTTGAAGAGAAGTTAAAG
GAGAACTTGGGAGCGGAATATAAAGCGACATTTCCGCAGCGAATGGAAAAACAGGACGAGGAATTTCTCAGGGTT
GGAGTCCAAAAATCCGGTGAATCAGCTGGAATTTGGATATACCTAAACGGCAGTGAGTTTCATTGGCTGGATAAT
GAGGAAGACATTCAAAGGGCGGTAACAAAAGTGATAGAAGAGTACAAAAAAAAGAGAGGATTTTTAAATGGTCCA
TATCTCACAGGAGATAGCTTTAGCAACGTAAAAAACAATCTTGTTTGTGCATTGGCCAATCGCGAAGACAATGAG
GAAGTGCTGAAGTATATACCGCATATCAGATACTACGACATGGTGGCGTACTTCTTGATTTTTATTACGGTGGAT
GGAGAATCGTATGTTAGAACCGTGGTAAACGAGGATTTAGACCGGTGGGAAGTGGAGCTGCAGGACGTGCTGGAA
ACGGCAAAGTCAAATACAGATCTCAATCCACCGGTGATTGAAATGGTTGAAATTAAGGACTGGAAGGTGATATCA
GCTCCAATAGGAAACACATTTGGTGAGATGCTCCAATCTATCAAAAGCCATCAGGAACAGAAGAACCCTATGTAT
GTGCTGTCAAATCAAAGTGGCCTGTATGGAGCCAGCAATATGATTGACAATCATCTGTTGGGACAGATTTCAGAG
AGCTGTAATGACAGCCTCCTTATACTTCCGGTAGACATTCATGAGATTATATTAATTCCGTCCAGAAAAAATAGA
ACCTCTATTGCGGATTGGAAAGCTGTGATGCATGCCTTAAATATTGAAAATAAAGGAGTGAAGCTTTCTGACCTT
GTATATTTGTTTGACCGGGCGGATAAGAAACTGCATGTAGCAAAAAATGATGATTTTCAGGCATAA
>3250057
ATGTTAGTCTGTGCCGGTTTTTTGGAAATGAGGAAAAATATGATACGACACGATAGCTTTTTCAAAAGACTGAAG
CGTGGTGGCTCGCTGTTTTTAGCGGTGTTACTTGCGGTTCAGTCTTTTTTTAATGTTGGATTTGGTATTATTTCA
GCAGATGCATCGGAGGGAGAAAACAGAGGAAGTACGTTTGCCTATTATGGCTCCCAGAATCTTTTAAAAGAGGGA
GCAAGTTTTCCCTTTTATGGAAAAAACCATAATCGGGTTGGCCTTTGGCCATATGGGATTACCAATGTAGAAGGC
GGCCATTCAGCACCTGGATACTGCCTGGAACCGAATAAGTCCATGCGTTCGGGCACTCCGGGAACGATTGTAACA
TATGACCTTGATACGGATGGTGACAACCTGCCCCTTGGGCTGACAAGAGAGGATGCGGAAATTTTATGGTACGCC
CTCTCATCATCAGGTAATTTTGAAGGAGGCATTTCGGGAAATGGGAAAATCGGCCAGGGACATTATATTTTGGGG
CAGGCAGCTACCTGGGCCATTATGTCAGGAAATTGGAATGGGTTAGATGATTTCCGTAGTCAGATGGAAGTCCTA
ATCGAGAACCTGAAAGATCCTATGCTTGCGGTATTGACAAGAGGAGCATTGGAACAATTTTTTAAACAGGTTAAT
GGAGCCGTGGAGGAAGGAGCCGTTCCACCCTTTGCATCTAAATTTCAAAGCCAGGCGCCGGTACATAAAATGAAA
GAAAACGGGGATGGGACCTATAGTATTACATTGGAGTTTGACGGTGATGATTGGAGGCAGTCCACGCTTGTTTAT
GATTTGCCAGAAGGGTGGAATGTGTCTCTGGAACATGGCAGAATCACATTTACATGTACGACAGGTAATCCCGAT
ATTGGCCTTGTGAGGGGACATTTTCAGGATGGCTCCCTGGGGGCTCAATACTGGGTTAAGCCAAATAGCTTTAAA
ATCTGGTATCCGGATGGTTGGAACGAGAGCAGTGCAGTAGATGGGAAACAGGCCATGATTACAATGGCAGGGAAA
CAGGAATCGTGGGAGGTTTGGCTGTCTTTTGGAAAAAGTACAACACACCGAGGGGAAGGAGACTATGAAATTCCA
TATACCCAGTACCTTCATGAGGAGACCTTTAAGCGGGATTATGTCATTGAACTGGAAAAGCAGTGTTCCGAGACA
GGGAAAACTTTGGAAAACTCCACCTTTGAAGTATTAGAAAAGTTTGAGTTTTCGCAGCTTGACGGGACAAATCTG
GAAAAAGACCAGTTCATGAAAATGGTGCCCACATCAGAAGGGAAATTTGAAGATTTAACTGTGTGCGATATGGGG
CTTGGTACAGATGCCAACGGACATTTTTCGCATTCGGATAAGAAGCTTTATAAATATGAGAAGACTTATTGTGGG
GGACACCCAGATCCAGTCATTCATTATGTGGATGGTGACTCTGACTCGGCCGATGAAGAAAATGAACGCCGGAAG
AAGAAAGCCTGGGATGCCTGGCAGGAGTGCGTGGACTGGTGTGAGGAAAATTGTGATTTCCATTCCATCGATGAG
GGCGTTGCCAGAGACTTAATGGAGGAGGACCGGGATGAGGCATGGAATACCTATATTCACCTGAAGCGTATTTAC
ACAGTCAGGGAAACAGACGCCAGAACCGGATATATCCTTCATGACCTGCACAATGATGATTTCTCCATTGAAATT
GTTGAATTTTCATCTTCTCAGTCAGAAGGAGAGGGTGCTATTACCGGCTATTACCCTGGCAATCGGGCAGTTTCG
GTTCGAGAGATGGAGGATGTGCCGCAGTTGTCAAAATCGGAGAAGGTTACAGATATTGTTCAGGCGGGAATGTCA
GATGAAGGGAATCAAACCAATGATTCAGTTACCGGACAGGAAAAAATACCTGAGGATAAAGGGAATGAACAAGAG
CCTGCTACTAAGGAACATGAGAACACAAAGAAACATGAATCGGAATCTTCCGGTGACACAGCAGAGAACGGCGAG
GAAGGGGAGAAGGAGGTGGAGGCCGGGTCAGAGGAAACAGCAGATACAACAGAAATAAAAGAGTCTGATGAGGCA
TCCCAGCCATCAATGGAAAACAGAGAGGAAACAGATGAAGAGGCCAGTCCTGAAGCTGAAACGCAGGATTCTGGA
AATGAAGCTGAATCAGTGGAAGAAGAGACCATAACAGATGACCTTCCTACCGTAGCAACCAGATCCCAGATAGGG
ATAGAAAAGGACGTGGCCACACCGTCAAATGCGTCCACCTATTTGGTTTCACGCCCGGCCTCTATAAAAGTTGGA
ACAGGCGGGCACTGGGAATGGGATGGTGTCCAGGAAGACAGTGAAGTGGCACCGATTGAGCAAGGCAGCTATCCG
TCCGGGTACATTGGTTACGCTTATCTGGTGAAGGATCACAGGACAGAAGGAGAGCTGCATATTAATAAGCGGGAT
AAAGAACTTTTTGAGCTGGAAGAGGACAGCTATGGAAAAGCCCAAGCAGATGCAACCTTAGAGGGCGCTGTATAT
GGACTATATGCGGCAGAGGACATCAGGCATCCAGATGGGAAAACCGGTGTTGTATTTTCTGCGGGGGAGTTGGTA
TCCATTGCCACAACGGATAAAAATGGTGATGCTTCATTCTTAACCATAACAGAAGTATCAGAAACCTCAAGAGAG
GTACCGAATCTCTATACGGGTAATGAAGTGCGGAATGGAAATGGCTGGATTGGCCGTCCGCTTATTTTAGGCAGT
TATTATATAGAAGAAATCTCACGCTCTGAAGGTTATGAACGTTCTGTAACCGGCAAGAACTTATCTGAGAGCAAT
CGAACAGGAAAGCCTATTGTCTTAACCGCGTCAGGGAGTGCCTATACAGATGGATTTACCCATAGTATTAATGAA
TGGTTTGAGGATTCCTATGATTTTACGGTTAAATATTATAAAACAAAGGGTTTCGATATTCTGCTTTCCGGTCTC
CCGGAGCGTGTCAAGGCATATGAGGTGACAAGAAAAGAGACTGCATCACAGGAGCAGGTGATAACCGGAACAGAG
TGCGTAGAGAAAAAAGATGCGCAGGGAAATATCCTGTACCGGACTGCCGAGGGTGGAGAATACAAGTTAGACGAA
ACCGGGAATAAGATTATAAAATCAGATGATAGCGGCAATCCGGTGTTGTCTGACCGGGCGCTCACCCAAACGGTT
TCGGCAGTGAACCGATTAAATGATTATATCTCATCCATTGAGCGGGAGGAACCGTCCGATTTGGATATGGAAGAA
TCAGAAGATATTGACGAGGATTATATTTTGTGGGAGACGTCATCGGCTCTTGCTCTTTCGGGGTATAAGAGTGGG
CTGTCTGATTACCCGTTTAAAAAACTCAAATTATCTGGAAAGACAAATGGGAAGATAATAGATGAGATTCTGACC
TTCTGCTCCTCGGAAAGTTTTTGGGATGCATATGATGTAGAGGCAGTATATGAGGAGGCGGGAACCTGGTATGCA
AGAATTCGTTATGGATATAAAGCATTGGCGAACCAGCCAGCCATTTATGAAAGCGGCAGTGGTCTTTTAGTAATC
CGAAAGGAATATGAGGGCGGGTTTTATTATGCAGTCTATGAAGAGGGACAGTATGATATGGATGGTTACCGCTTC
ACAGTAGAGAAAAAGGAACTGGACCTGGAGGCTTTGGGGCAAGACGAAATTCTGTTAAAGACGGTTTATGCACCT
GTCTATGAGACCTATGCGACTGGGGAGTTCATATTGGACAGTGAAGGACAGAAAATCCCGCAGATGGAGGCGGTT
CCAATCTATACCAGCCAGGAGGTGTTTTCCTATGAGGAAATTCTGACCCCTGTAAAGACAACCGCAGTGGAGGGC
CAGGTGAGCATCCATTTGGATACGGACGAGGAATTTACAGAGGGGGAAAAACATGAGAGAACTTACCGGGTAGTG
ACAGAACAGAAAATAACAGAATATACAGCAACCGTGAATGTCATGACGACCAAACCTGCACAGAAGAGTGGCTCT
TATCTAAAGTTCCCTGTATTATTCTATCCGGGCCAGTATGAAATCTATGAAGATAACGGCACACGGAAAGAGCCG
GTTATTATGCTGGAGCGGGTGATTAAGCAGGCAATCGAGGTGAAAAAGGATATTGCTCTGGACTCCTATGAGCAT
AATACCTATGAGATTCACCGTGACCCGTTTACCGTTCTTTTTGGAGGGTATAACGGAACACAGGAGACGAAGACG
CTTCCCGGCTTTTTCTTTAAACTGTATCTGCGTTCTGATTTAGAGAAAACAGACAAGCTTATGAAAAAGGAAGAT
GGCAGTTATGACTATGTAACATTTTTCAAGGAAAATCCCGATGCTGCGTCTGAGCTTGCAATTGAGTGGGATTTG
GAAAAGTATGATGCAGACGGGGATATGACAACCGTACATGCAAACCGGGGCGGCGGGAAGGACGACTACTGGGGA
CAAAGCAGGATGCTTCCTTACGGTACATATGTTTTGGTGGAGCAGCAGCCAACAGGCATCCCACAAAAACATTAT
GAGATTGATGCGCCTCAGGAGGTAGAGATTCCTTTTGTTCCACAGATAGATGCCGATGGTACAGTACATGATAAA
ATCCCATCTAAGGAATATCTTTACGACTCTGCCATGACACCGGAGGAATTGACTGAGCGTTATCAGATTCGATTT
AATGAAGAAACCCATATCATATATGCGCATAATAATGACGGCGATTTTGAAGTGTTTAAGTATGGTCTGGAGCCG
GACAGCAGAAGGGATTGCCAGAATGAGACAGTAGCGAGGTATTATCATTACGGATCTATCAGCGAGGATGCTGGA
AGTGCAGACCAGGTGTATTATGAAACTTATTACGACCGGGATGGAACGATAGCGGATTATGGTGTAACAATGAAT
GGAGTGGTTACCATGACCGGAAAGTCTACTGCCGTGGACCGGATGTACGCGAAAGCACTTGTACCCTGGAGTGTG
TTAGATCCCAGATATGGAGAAGTAATCAACGATGACGGCGATATCGGAAACCGGGAAGCTGGCCTGGAGACAGAT
GGAACCTTTAATTTTATTTCCTTTGCAGTCAAGGATTTTGAGAATGAGTTTTACAGCTCCAGACTCAGGATTGAG
AAATTGGATTCTGAAACTGGAGAAAATATTCTGCATGAAGGAGCACTCTTTAAGATTTATGCAGCCAAAAGGGAC
GTGATTGGAAATGGTGCTTCCGGTGTGACAGGAAGCGGGGATATCCTGTTTAATGAGGAGGGAACACCTCTCTAT
GAGGAACGTGAACAGATTTTCATGCAGGACGACACCGGAGCGGAAGTAGGGGTTTTTAAGGCTTACACCACAATC
CGGGACGGAGAAGTTGAAGGAGAGGACGGAAGCCTGCATACAGAGAAGCAGTGCGTGGGGTATCTGGAAACGTAT
CAGCCGTTAGGCGCCGGCGCTTATGTTCTGGTGGAGGTAGAGGCACCGGATGGATATGTGAAGTCGAAACCTATT
GCATTTACGGTATACAGCGACAAGGTGGAATATTATGAAGACGGCAATCAGGAGAAAAGGACGCAGGCGGTAAAA
TACCAGTACATGCGTCCCATTGGCGCTGACGGGAAAACCGTTGTAGAGGATATGCACCAGATTATTGTAAAGAAT
GCACCTACACATATAGAAATACATAAGCTGGAAAACCGTGCGGATGCCGTCACATACCGTGTAGAGGGAGATGAA
AAGCAGCTAAATAACCGCGGGGACGTGGATTTACAGTATAAACCCAATGGGGAATTTGCCGGGTTTGGTTATGTG
ACAAAACGCCTTGAAAACGGTCAGGAGAAGATATATGTGGAGAATGCAACACTGACATTGTATGAGGGGTTGGAA
GTAAAGCAGACTGGGGAACACGAGTATGAGAAAGTCAAGGTGAAGAGAAATCTATTTGGCTCAGTAACGGGAATC
CAGGCGTATGATACCGGCGTGGATACCGATATAAGACAGACTGGGACAAACGCAGCAGGGCAGGCGGAGTGGGAT
ATTACAGAAGAGGATAATCCACCCGTGGACATATGGTATTTTGACTTAAAATATGATCCCACAGAACTTGATGAA
CAGACCGGCATCCTCTATGGACTGGATGACTGGGGGAACCGTCTCTGTATGTTGGATTCAGAAACAGGTATGGCT
TATGTGACAGATAAAACCGGCGCAGTGATTGTCTGGCCGCTTGATGAAAATGGGGATAAAATCATTTCCCAATCC
GTAGAAGTATATACCAACGGGGAAGGAAATTCATCTATCAACATGGATTTGCAGCCAGTACCAGATGAAAACGGG
CTTCCTATCTATTATAAGGATGGCGGTGTTATATGGATTGAGAATGAATGGGTGACGGACCATGGTGCCTGTGAA
ATTGCAAGAGTAAGGCAGGGCGCCTATATCCTGGAGGAGACAGCTGCCCCGTTGTCAGACGGTTATGTACAGTCC
GCATCAGTCGGTGTGATTGTCCGTGATGTAAGCGAGAAGCAGTCCTATGTGATGGAGGATGATTACACAAAGATT
GAGGTATCAAAGCTTGATATGACTTCCAGAAAAGAGATTGAGGGCGCGGTTTTAACACTCTACGAAGCATACCGC
GTCTATGATTATTCCGTCCGGGGGTGGCATTTGGAAATTCTCCGGGATATGGAGGACAAACCGATTGTAGCGGAA
CGATGGGTGTCAGAAGGAAACGTTCCACACTGGATTGACCATATCATGCCGGGAGATTATATTCTTCAGGAAACA
AGAGTACCAACAAAGGCCGGCTATGTGACGGCGGAGGATGTGGAAGTCACCATATTGGAAACCAGTGAAGTACAA
GGCTATGTAATGGAAGATGATCATACAGCGGTAGAGGTTTTAAAGCTGGACTCCCGGACCGGGGCTGTGATGGAT
AATCTTCACCGGGCCACACTGGCTTTATACGAAGCCCAGGTAGATGAGAATGGGGAAGTGCAGTATAGAGATGAT
GGAACCATACTCTATCATCAGGAAAAGAAGGTGTATGAATGGCAGACAGATGATGGAAGTGATGTGAGAAAAACG
GCCCATCAGGTTACCATACCGGGTGGACACAGCTACACGGCCTATGATTATGAGATAGAGCAGGTTCCGGGAACC
AGTCAGGCGGTTTGCTATATCACGGAGACAGGAGCCATGCGTTTTGAGTATCTTCCGGTGGGGAAATATGTGTTG
GTAGAAGAGCAGGCTCCTTACGGCTATACAGTGGCAGCACCGGTTTATGTACCAGTCCTTGATGTGGGTTCTAAG
GAACGTGTGCAGACCATTACCATGACAGACGAACCGATTCAGGTACTCCTGACAAAAGTGAATGTGACAGGTGGA
AAAGAAATTTCCGGAGCCACAGTTGCCGTCTACCGTGCAAAGGAAGATGGTACACTTGCCAAACACCAAATGAAA
GACAAAAACGGGAATCTGTTGTATGTAACCGATACGGACGGGAATCTGTTGCAGGATGAAGACGGGAACCATATT
CCGGCTATGGAGTACGAAGAGGCGTATCTGGTGGAACGCTGGATTTCCGGCTCAGACGGAACATACACGAAGCGG
GACCAGAAAGAAGGCAGGATACCGGAAGGTTATGAAATCGGGGATTTAAGGCTTCATGAGTTAAGTCAGCCAGCC
GCAGGGAATTACTATTTTGTTGAGGAACAGTCTCCCTTTGGATATGTGAGGGCTGCAGAACTGCCGTTTGCGATT
GTTGATACACTGGATATTCAAAAGATTGAACTGGTAAATGAACTGATTCTGGGCCAGGTTGAGATTATCAAGACC
GATAAAAGGAATCCGGAACAAGTTTTATCAGGTGCAAGGTTCCGATTGTCCAATCTGGATACCAATGTGGCGACC
ATTCTGATAACGGATTCCGATGGTCGGGCAGTCAGCTCCCCGGTCCCCATTGGAGGGATAGGAACGGATGGGGCG
GTAAGCCTCTATCATTTCCGGATACAGGAGGTGGAAGCGCCTGACGGTTATCTGCTTGACCCGACAGTCCACGAC
TTCCAGTTTAATATAAAGACAGACCAGTACCAGACGCTCACCTATCAGTATGAAGCAGCGGATAGCCCTAATAGG
GTTATCATTTCTAAAAAGCAGCTTACAACAAAGGAGGAACTGCCGGGCGCCAGCTTAGAGGTCCGCAGAGTCACA
GAGATGGTTGACAAGGACGGAACGGTTATACGGATTGACGGTGATGTGATTGAAAGCTGGATTTCCACGGAGGTC
CCGCACGAACTGGAATGCCTGACAGAGGGAATGTATGTCCTAATCGAGACACGGGCACCGGAAGGATATATAGAA
GCAGAGAAAGTGTACTTTACAATATCCGGGAACATGACAGTGGATGACATGCCTATGGTGGAGATGTTTGATGAT
GACACCAAGATTGAAATCAGAAAGGTGGACAGTGAAAACGGAAATCCTTTAAAGGGTGCGAAGATGCAGCTGATT
TTGGAAGCGTCCGGGGAAATCATTAAGGAATGGATTACAGATGAAACCGGAGCGATTCAGTTCTTCGGTCTCCCG
GCAGGCGTGTACTTAGTGAAAGAGGTAGAGGCTCCGGAAGGATACCAGATACCGGAAGGTCCCATGCGGATTACC
GTGACCAAAGATTATAAGCAACAGACGTTTGTAATGGAGAACCGGATGACCGAATTGATGATTGACAAACTGGAC
GAGGAGACCAGGGAACCGGTAACAGGAGCAGTGCTGCAGCTTACAGATGGGGAAGGAAATCAGGTGGCGAAATGG
GTTACCACTGGGGAACCGGAACTAATCCGCGGCTTAAAGGCAGGCTGGTATATACTGGAGGAAATCCAGGCGGCA
GACGGCTATCTGCTTTTAAAGGAACCAGTGAGAATCGAGATTACGGAGAAAGCAGGAATACAAACCGTTACCATT
ACAAACCGGAAACTTGAAGTGGAAGTGGCAAAAACAGATAAAGAGACCGGAGAAAACCTGGCGGGAGCAAGACTT
CATTTGATTCGAGATGCCGATGGCATGGTATTGAAAGAATGGGTAAGCGGGAAGATACCTGAAATTTTTAAAGGA
CTTTCTGTCGGAGAATATACAATCAGGGAATTGACGGCCCCAGAGGGCTATGCAGTAACAGGGCGTTTAAACTTT
TGCGTGAGCGGAACCGAGGAAAAGCAGGAGATTAACCTGGTAAATGAGAAGATTGTGGTGGAACTCCAGAAGACG
GACACCTTGGAAGGCAGCCCAGTGGAAGGGGCGGTTCTTCAGCTTCTAAAGGCTGCGGGGACAAACGAGGAGACT
GTAATGAAAGAATGGGTGTCAGGAAAGGAGCCGCTTATTTTAACAGGAATACCGTCTGGTATCTATACAATAAGG
GAAACACAGGCACCGGAAGGATATGTCCCAATGGAGGATATGAAAATTGAGATCCTTCCTAATCAGACCATGCAG
CATTTTGATATTAAAAACCAGCCAATTCAGATAGAGATAGGAAAGGTTAGCGGAGAAACAGGAAAACTGCTGGGC
GGCGCTGTTCTTCAACTGGTAAGAGATTCGGACGGTACAGTAATCCGGAAATGGACATCCAGGGACGGTGAGGCT
GAACACTTTAAAAACCTTGCGGGCGGTACGTATATAATACGTGAGGTGAAGGCTCCCTCCGGGTATGAGAAAATG
GAGCCGCAGGAAATAGAAATAAAAGATATTGAGGCGATACAGGAATTTGTGGTCAAGAACTATAAAATCACCCAT
TCAGGTGGCGGCGGTGGCAGCACTCCGAATCACCCGAGACCCTCAGCGGAGTATATGGAACTGTTTAAGATTGAT
GGAAGAACTGGCCAGAAGCTTGCAGGGGCAAAGTTTACCGTTTACAATCCAGACGGCAGTGTCTATGCGGAAGGA
TTTACAAATGCAGCAGGAACATTCCGGTTTAAGAAACCTTTAAAAGGAGCCTATACATTTAAAGAGACGGAGGCA
CCAGAGGGATACTATCTAAATGAGGTACTGCATGCGTTTGCAGTAACTGAAGATGGGACCATAGAAGGGGATACG
ACTATGGAAAACTACAGTAAAACAGAAATGATAATCTCTAAAGTGGATGTAACAACAGCAGAGGAACTGCTTGGC
GCCGAGATCGAGGTCACAGACCAGGACGGAAACCATATCTTCTCCGGCATTAGTGATGAAAAGGGAAAAGTATAT
TTTCCGGTCCCGTCTCCGGGGGAATATCATTTCAGGGAACTGACAGCACCGGAAGGGTATGACAGAAATGAGACG
GTCTTTTCATTCACGGTGTTTGAGGACGGCAGTATAATTGGCGACAATACCATTACCGATCAGAAACATTACGGA
ACAATCACGGCCAATTATGAGACAAATAGAAGGGGAGAGGGTGATTTGACGGTCGGTGAACTTAATCATGCACCA
AAGACAGGAGATACCAGCTATTTTGTAGGGGTGTTCATGGCATGGCTTGCATCCGTAGTAAGCCTGTTAATTGTT
TCCCTCTCAAAGTGGAGGAAAAAACAAAAGAAATCAAAAACAGGGATAAAGGCTGGCATGTTTCTATTATTGGCA
ATAATGGTAGTTAGTACGCCGGTGATGGAGTCTCAGGCAGCGGAAGATGTAATTGAAAATATATATGAGGAACAC
CAGTATACAACGGAAAACCCCGATTCTAATGAAGCAGAAAAGCTGTTTGAAAAAGAAATTGAGAGAGACGGAAAA
AAATACCGTCTCTCAGAAATCCGGACAGAAGTGCTGGAAGAACAGGGGAAAAGGAGTTCTGGAAATTCTTTTGAA
ATCGCAACAAGCCCGTTTATTGATGGAAAAGTGGAGGTTAGGCCGAAAGAACAGATAACCCGGAATGGAATTACT
TATCATTTGGTAAAATCCCAGAAGGAAGCAGCCGTTATACGGGCTCATGAGGTACCGGTAAGTGAGGAAGTCTTA
TATGAAGCAGTGGAAGCCAAGGACATGATACCGTCAAAAATCAAGACGACGGTAACAGATGAGAAAACCGGGCAG
ATGATGGAGACTATTGCAAAAATTTCTGACCAGACATTTGGCGGGGAGCGCCTGGACGATACATTTTCCTTTTCA
GCTGTGTTCCATGAATATGGGCTGGACGGCTATTGGATTGGGGACCAGGTTTTTAAACTGAGTGGGACTGAACCA
GACTTCACCGGATATGAAGGGCAGCTCCTTTCTCTGATTGAGGCTGATGCTGAGCATTATACCATAGAAGCAGCA
AGGTGGGATGGGGAAGCTTACACAGATGAAGCAGGTATCCTATGCCGTAGGGTTGTTATAACTGGTACCAAGAAG
GTGAGTGACTGCACAGTGATGTATGAGGGAAGTGCCTATTTTCCGGAGGAGGAAGGCGTACGCCTGATTTCTACC
TATGATAGCGGGGAAGAAGAGGTAGAGCAATACACCATGAAAGCAACGGGGGTTTACATACCTAAAAAGAACCAT
GGAGCAGCGGTCGCCACTGTGATTGGTGTTACCAGTGTCGGTGCAGGTACTGCAGGATATACCTATTATCGGAAA
AAGAAGCAGAATCAAAACGTATAA
>3253773
ATGAAAAGGATACTATCCAGTGCCATACAAGTATTTAAGCAAATCAAAAGCGACCCTATGATGTTTGCGGCTTGC
TTCACCCCTTTTATTATGGGAGCTTTAATCAAATTTGGTATTCCATTTTTTGAAAGAATAACAAAGTTTTCTTTA
CAAGGATACTATCCGATTTTTGATTTATTGCTTTCTATCATGGCTCCTGTACTGCTTTGTTTTGCATTTGCCATG
ATTACGTTAGAGGAAATTGATGATAAAGTATCGCGGTACTTTTCAATTACCCCTCTTGGTAAGGCGGGGTATCTC
TTTACAAGGTTGGTAGTACCCGCAATTATTTCAGCGGTCATTGCTTTTATCGTACTTTTGCTTTTCTCATTAGAA
AAGCTACCCACTAGAATGATGATTGGTTTAGCACTTCTCGGTTCGGTACAGGCAATCATTGTTTCACTTATGATT
ATTACCTTATCCGGTAATAAATTAGAGGGTATGGCTGTAACAAAACTTTCTGCACTTACACTGTTAGGCATTCCA
GTTCCTTTCTTTATAGATAGTTACTACCAGTTCGCGGTTGGCTTCCTCCCATCATTTTGGGTAGCAAAAGCCGTA
CAGAATGAAGCAGTTCTTTATTTTCCCACAGCATTGGTAGTAGCTTTGATCTGGTACTACTTTCTTATAAAACGT
CTGTTTCGGAAGCTGGCAGGATAA
>3646621
TTGCAGAAAAAAGAAGATGGAATCATTTTGAAAAAAATATTAATTGCTTTAATTAAGTTTTATAGAAAATATCTT
TCTCCGATGAAAACGACCAAGTGTCCATATTGCCCGACCTGTTCTTTATATGGGTTGGAGGCAGTTGAAAAGTAC
GGAGCTCTAAAAGGGGGAGCACTTGCTTTATGGAGAATCTTAAGATGTAATCCTTTTTCAAAAGGTGGATATGAT
CCAGTTCCATAG
>3793132
ATGGTTACTGTAAATAATTCTGACTATTATAACAACATTCCAGCTGGCAGGGATTTAAACAAGCTACCCAATAAT
TCCAGCGCCGGTACAACTGTAGGATATCAGTATGATGGACTTACAGAGGAAGATCGTTTCGTACAAAAAGTTTTG
CGGGAGCATTATGATAAAATGTACAAAGAAAATATGTCGCTTTCTGATCCAATGGCATATGTCATATCGAAATAT
TGTGATGTGACTTCACCTAACTTTTGCTCATATATGACAGAAGACCAACGTTCCATAGCTTACCGTACAGAGAAA
AGAATGTTACAATCCGGAGGAAAACCTGTGGGTGGATTTGCACGGTATGATTATGCATTAAGGAATTACAAGGAT
GTATATACAGGTGGTTCAAGAAGTGTTGGTTATGTACGCAATACTGACAGGGAAAAACAGCATGCCAGAAGTGTT
GTAAATCAGCAAATTTCAAATCTACTTTCAGAGAATGGGATATCAATATCAAAACAGGCAATTTGGTATTTTCTA
TTGACCCATATACATATCAACTAA
>3815768
ATGTTTATTACCTGTTTGGACCTGGAAGGAGTATTAGTACCGGAAATCTGGATCGCATTCGCTGAGGCCAGCGGC
ATCCCGGAGTTAAAGCGCACCACCCGCGATGAGCCGGACTATGACAAGCTGATGAAATGGCGTCTCGGAATTTTA
AAGGAGCACGGACTTGGCTTGAAGGAGATCCAGGAAACCATCGAGAAGATCGACCCGATGCCGGGAGCAAGAGCG
TTCTTAGATGAGCTCCGGGAGCTGGGACAGGTAATCATCATCAGCGATACCTTCACCCAGTTCGCAAAGCCACTC
ATGAAAAAGCTGGGCTGGCCAACCATTTTCTGCAACGAGCTGGAAGTAGCAGAGGATGGTGAGATCACCGGATTC
AGAATGCGCATTGAGCAGTCCAAGCTCAGTACCGTCAAGGCACTGCAGTCCATCGGCTTTGAGACCATTGCCAGC
GGCGACAGCTACAATGACCTTGGCATGATCCGCGCCAGCAAGGCCGGCTTCCTCTTTAAGAGCACCGATGAGATC
AAGAACGACAATCCGGATCTTCCGGCGTACGAGACCTATGAGGAGCTGCTGGCAGCGATCAAGGCAGCAGTATAA
>4097912
ATGATATCCACAGTCACAAAAGCCCATGCACAGCAGGAAATCCTGCTCCTCGCCATGAAGGCCGGGCAGATCCAG
CTGGAAAACGGAGCGGAAATCTTTCGCGTGGAAGATACCATCATGCATATCTGCCGCGCCTACGGACTTCATTCT
GTCCATATTTTCGTGCTGAGTAACGGTATTTTTCTAAGCTGCGGAGATGAGACGGAACCCCTGTTTGCCAAGGTT
TTGCAGGTGCCTGTCAACAATACCAACCTGCGCAGAGTTGCGGAAGTAAACCAGCTATCAAGGCGCATTGAGGAA
GAAGGGCTTTCCCCG
>4136092
TGGGCTGTTGCGACCGCGCATCGGAAGAATGCTTACGAAAGCAAGATCGGAACCGCGGAAGAAAAAGCCAGGGAA
ATAATAGATGAAGCGTTAAAGACGGCAGAGACAAAGAAGCGAGAAGCTCTCCTGGAGGCGAAGGAAGAGTCCTTA
AAGACTAAGAATGAGCTGGATAAAGAGACAAAGGAAAGAAGAGCTGAACTTCAGCGCTATGAACGACGTGTGCTG
AGCAAAGAAGAAAACTTAGACAAAAAAACAGAGAACCTTGAACGGCGGGAAGCCGGGCTTGCATCCCGTGAGGAA
GCCTTGAACAAGCGTAATGGTGAGGTTGAGGCCCTTTACGAAAAAGGGATACAGGAACTGGAGCGTATTTCCGGT
TTAACCTCCGAACAGGCAAAAGAGTATCTGCTCAGATCTGTTGAGGCGGAGGTCAAGCATGACACTGCCAAGATG
ATCAAGGATCTGGAGAACAAGGCAAAAGAAGAAGCTGACAAAAAGGCAAAGGAGTATGTGGTTACTGCGATTCAG
AGATGTGCTGCAGACCATGTGGCTGAAACTACCGTATCTGTAGTACAGCTTCCGAACGATGAAATGAAGGGACGC
ATCATTGGCCGTGAGGGACGTAACATCCGTACCCTTGAGACTATGACTGGTGTG

The specific embodiment of the present invention has been described in detail, and skilled in the art will understand the same. According to the published guidance, modifications and replacement of those details can be performed. These changes are within the scope of protection of the present invention. The full scope of the present invention is given by the appended claims and any of its equivalents.

In the description, the term “one embodiment”, “some embodiments”, “schematic embodiment”, “example”, “specific examples” or “some examples” means the specific features, structures, materials or characteristics are included by at least one embodiment or example in the present invention. In the description, the schematic representation of the terms above does not necessarily mean the same embodiment or example. Moreover, the description of the specific features, structure, materials, or characteristics can be combined with in any one or more embodiments or samples in a suitable way.

What is claimed is:

1. A method to determine biomarkers related to an abnormal condition in a subject comprising:
sequencing nucleic acid samples from a first and a second subject in order to obtain multiple sequences respectively consisting of the first and the second sequencing results, wherein the first subject is in the abnormal condition; and the second subject is not in the abnormal condition; and the nucleic acid samples from the first and the second subject are both isolated from the samples of the same type; and the first and the second subject belong to the same species; and
determining the biomarkers related to the abnormal condition in the subject based on the difference between the first and the second sequencing results.

2. The method of claim 1, wherein the abnormal condition is a disease.

3. The method of claim 1, wherein the disease is selected from at least one of neoplastic diseases, autoimmune diseases, genetic diseases and metabolic diseases.

4. The method of claim 1, wherein the abnormal condition is diabetes.

5. The method of claim 1, wherein the first and the second subject are human.

6. The method of claim 1, wherein the nucleic acid samples from the first and the second subject are isolated from excreta of the first and the second subject respectively.

7. The method of claim 1, wherein sequencing nucleic acid samples from the first and the second subject is conducted by means of second-generation sequencing method or third-generation sequencing method.

8. The method of claim 1, wherein the sequencing step is conducted by means of at least one apparatus selected from Hiseq 2000, SOLID, 454, and True Single Molecule Sequencing.

9. The method of claim 1, wherein determining the biomarkers related to the abnormal condition is based on the difference between the first and the second sequencing results further comprises:
aligning the first and the second sequencing results against a reference gene catalogue; and
determining relative abundance of gene respectively in the nucleic acid samples from the first and the second subject based on the alignment result; and
conducting statistical tests on the relative abundance of gene in the nucleic acid samples from the first and the second subject; and
determining gene markers which are significantly different between the nucleic acid samples from the first and the second subject based on their relative abundances,
optionally, after obtaining the relative abundances, using the Poisson distribution to conduct the statistical test on accuracy of the relative abundances.

10. The method of claim 9, wherein, before aligning the first and the second sequencing results against reference gene catalogue, a step of filtering is used to remove contamination sequence, wherein the contamination sequence is at least one sequence from adapter sequence, low quality sequence, and host genome sequence.

11. The method of claim 9, wherein the step of aligning is conducted by means of at least one of SOAP 2 and MAQ, which aligns the first and the second sequencing results against a reference gene catalogue, or against human gut microbial flora non-redundant gene catalogue.

12. The method of claim 9 further comprises: performing de novo assembly and metagenomic gene prediction on high quality reads from the first and the second sequencing results, wherein the genes not matched with reference gene catalogue are defined as new genes; and integrating the new genes with the reference gene catalogue to obtain an updated gene catalogue; and conducting taxonomic assignment and functional annotation.

13. The method of claim 12, wherein taxonomic assignment is performed by aligning every gene of reference gene catalogue against IMG database.

14. The method of claim 13, wherein aligning every gene of the reference gene catalogue against IMG database is conducted by BLASTP method to determine taxonomic assignment of the gene, using the 85% identity as the threshold for genus assignment and another threshold of 80% of the alignment coverage, for each genes, the highest scoring hit(s) above these two thresholds was chosen for the genus assignment, and for the taxonomic assignment at the phylum level, the 65% identity was used instead.

15. The method of claim 12, wherein functional annotation is performed by aligning putative amino acid sequences, which have been translated from the gene catalogue, against the proteins/domains in eggNOG or KEGG database.

16. The method of claim 15, wherein aligning putative amino acid sequences, which have been translated from the gene catalogue, against the proteins/domains in eggNOG or KEGG database is conducted by BLASTP method to determine functional annotation of the gene, according to functions whose E-Value is less than 1e-5.

17. The method of claim 9, wherein the relative abundances comprise species and functions relative abundances, and the reference gene catalogue comprises taxonomic assignment and functional annotation, determining the biomarkers related to the abnormal condition based on the difference between the first and the second sequencing results further includes:
aligning the first and the second sequencing results against the reference gene catalogue; and
determining species and functions relative abundances of gene respectively in the nucleic acid samples from the first and the second subject based on the alignment result; and
conducting statistical tests on the species and functions relative abundances of gene in the nucleic acid samples from the first and the second subject; and
determining species and functions markers respectively which are significantly different between the nucleic acid samples from the first and the second subject based on their relative abundances,

18. The method of claim 9, wherein the statistical test is conducted by at least one of Student T test and Wilcox rank sum test.

19. The method of claim 9 further comprises enterotypes identification.

20. The method of claim 9 further comprises clustering the gene markers and advanced assembling to construct organisms genome associated with the abnormal condition, by Identification of Metagenomic Linkage Group (MLG).

21. The method of claim 9 further comprises steps to validate the biomarkers.

22. A system to determine biomarkers of an abnormal condition in a subject comprising:
sequencing apparatus, which is adapted to sequence nucleic acid samples from the first and the second subject in order to obtain multiple sequences respectively consisting of the first and the second sequencing results, wherein the first subject is in an abnormal condition; and the second subject is not in the abnormal condition; and the nucleic acid samples from the first and the second subject are both isolated from the samples of the same type; and the first and the second subject belong to the same species; and
analytical apparatus, which is connected to a sequencing apparatus, and adapted to determine the biomarkers of the abnormal condition in the subject based on the difference between the first and the second sequencing results.

23. The system of claim 22 further comprises nucleic acid sample isolation apparatus, which is connected to the sequencing apparatus, and is adapted to isolate nucleic acid sample from the subjects.

24. The system of claim 23, wherein the sequencing apparatus is adapted to carry out second-generation sequencing method or third-generation sequencing method.

25. The system of claim 23, wherein the sequencing apparatus is adapted to carry out at least one apparatus selected from Hiseq 2000, SOLID, 454, and True Single Molecule Sequencing.

26. The system of claim 22, wherein the analytical apparatus further comprises:
means for alignment, which is adapted to align the first and the second sequencing results against reference gene catalogue; and
means for determining relative abundance, which is connected to the means for alignment and adapted to determine relative abundance of gene respectively in the nucleic acid samples from the first and the second subject based on the alignment result; and
means for conducting statistical tests, which is connected to the means for determining relative abundance and adapted to conduct statistical tests on the relative abundance of gene in the nucleic acid samples from the first and the second subject; and
means for determining markers, which is connected to the means for conducting statistical tests and adapted to determine gene markers which are significantly different between the nucleic acid samples from the first and the second subject based on their relative abundances.

27. The system of claim 26, wherein the analytical apparatus further comprises:
means for filtering, which is connected to the means for alignment and a step of filtering is provided to remove contamination sequence before aligning the first and the second sequencing results against reference gene catalogue, and
the contamination sequence is at least one sequence from adapter sequence, low quality sequence, host genome sequence.

28. The system of claim 26, wherein the means for alignment is at least one of SOAP 2 and MAQ, which aligns the first and the second sequencing results against reference gene catalogue, or the human non-redundant gene catalogue.

29. The system of claim 26, wherein the relative abundances comprise species and functions relative abundances, and reference gene catalogue comprises taxonomic assignment and functional annotation, further comprises:
the means for determining relative abundance, which is adapted to determine species and functions relative abundances of gene respectively in the nucleic acid samples from the first and the second subject based on the alignment result; and
the means for conducting statistical tests, which is adapted to conduct statistical tests on the species and functions relative abundances of gene in the nucleic acid samples from the first and the second subject; and
the means for determining markers, which is adapted to determine species and functions markers which are significantly different between the nucleic acid samples from the first and the second subject based on their relative abundances.

30. The system of claim 26, wherein the means for conducting statistical tests is conducted by at least one of Student T test and Wilcox rank sum test.

31. The system of claim 26 further comprises a genome assembling apparatus, which is adapted to cluster the gene markers and advanced assemble to construct organisms genome associated with the abnormal condition, or by Identification of Metagenomic Linkage Group (MLG).

32. The method of claim 9 further comprises assessing the effect of each covariate as enterotype, T2D, age, gender and BMI or use Permutational Multivariate Analysis Of Variance method.

33. The method of claim 9 further comprises correcting population stratifications of the data, wherein adjust the gene relative profile by using EIGENSTRAT method in order to remove the covariate effect.
